SEQUENCE LISTING (33 sequences)

SEQ ID NO: 1 - protein, 15 aa, synthetic construct
  REGION 1..15: note = synthetic polypeptide
PVSKARMATGALAQA

SEQ ID NO: 2 - protein, 15 aa, synthetic construct
  REGION 1..15: note = synthetic polypeptide
PVSKMRMATGALAQA

SEQ ID NO: 3 - protein, 15 aa, synthetic construct
  REGION 1..15: note = synthetic polypeptide
PVSKMRMATPLLMQA

SEQ ID NO: 4 - protein, 15 aa, synthetic construct
  REGION 1..15: note = synthetic polypeptide
PVSQMRMATPLLMRP

SEQ ID NO: 5 - protein, 16 aa, synthetic construct
  REGION 1..16: note = synthetic polypeptide
ISQVHAAHAEINEAGR

SEQ ID NO: 6 - protein, 15 aa, synthetic construct
  REGION 1..15: note = synthetic polypeptide
PVSQARMATGALARP

SEQ ID NO: 7 - protein, 14 aa, synthetic construct
  REGION 1..14: note = synthetic polypeptide
LPLKMLNIPSINVH

SEQ ID NO: 8 - protein, 15 aa, synthetic construct
  REGION 1..15: note = synthetic polypeptide
HTYTIDWTKDAVTWS

SEQ ID NO: 9 - protein, 13 aa, synthetic construct
  REGION 1..13: note = synthetic polypeptide
PKYVKQNTLKLAT

SEQ ID NO: 10 - protein, 15 aa, synthetic construct
  REGION 1..15: note = synthetic polypeptide
EPDVYYTSAFVFPTK

SEQ ID NO: 11 - protein, 16 aa, synthetic construct
  REGION 1..16: note = synthetic polypeptide
LGLLYTVKMYPNLNDL

SEQ ID NO: 12 - protein, 15 aa, synthetic construct
  REGION 1..15: note = synthetic polypeptide
SPILRFLYANVGEEA

SEQ ID NO: 13 - protein, 15 aa, synthetic construct
  REGION 1..15: note = synthetic polypeptide
YFESFVREFVATART

SEQ ID NO: 14 - protein, 15 aa, synthetic construct
  REGION 1..15: note = synthetic polypeptide
IVYSLVTTISSLSRI

SEQ ID NO: 15 - protein, 15 aa, synthetic construct
  REGION 1..15: note = synthetic polypeptide
DITYKVHLATPINSR

SEQ ID NO: 16 - protein, 21 aa, synthetic construct
  REGION 1..21: note = synthetic polypeptide
MEVGWYRSPFSRVVHLYRNGK

SEQ ID NO: 17 - protein, 16 aa, synthetic construct
  REGION 1..16: note = synthetic polypeptide
GGGGSLVPRGSGGGGS

SEQ ID NO: 18 - protein, 17 aa, synthetic construct
  REGION 1..17: note = synthetic polypeptide
GGGGSENLYFQGGGGGS

SEQ ID NO: 19 - protein, 15 aa, synthetic construct
  REGION 1..15: note = synthetic polypeptide
LPLQPFQLAFGHLVN

SEQ ID NO: 20 - protein, 16 aa, synthetic construct
  REGION 1..16: note = synthetic polypeptide
LQPFQLAFGHLVNRQV

SEQ ID NO: 21 - protein, 16 aa, synthetic construct
  REGION 1..16: note = synthetic polypeptide
FQLAFGHLVNRQVFRQ

SEQ ID NO: 22 - protein, 15 aa, synthetic construct
  REGION 1..15: note = synthetic polypeptide
FGHLVNRQVFRQGPQ

SEQ ID NO: 23 - protein, 15 aa, synthetic construct
  REGION 1..15: note = synthetic polypeptide
HYKYMCNSSCMGVMN

SEQ ID NO: 24 - protein, 15 aa, synthetic construct
  REGION 1..15: note = synthetic polypeptide
YMCNSSCMGVMNRRP

SEQ ID NO: 25 - protein, 15 aa, synthetic construct
  REGION 1..15: note = synthetic polypeptide
NSSCMGVMNRRPILT

SEQ ID NO: 26 - protein, 15 aa, synthetic construct
  REGION 1..15: note = synthetic polypeptide
CMGVMNRRPILTIIT

SEQ ID NO: 27 - protein, 119 aa, synthetic construct
  REGION 1..119: note = synthetic polypeptide
DIQMTQSPSSLSASVGDRVTITCRASQGIRNYLAAWYQQKPGKAPKLLIYAASTLQSGVPSRFSGSGSGTDFTLTISSLQPEDVATYYCQRYNRAPYTFGQGTKVEIKRTVAAPSVFIF

SEQ ID NO: 28 - protein, 129 aa, synthetic construct
  REGION 1..129: note = synthetic polypeptide
EVQLVESGGGLVQPGRSLRLSCAASGFTFDDYAMHWVRQAPGKGLEWVSAITWNSGHIDDYADSVEGRFTISRDNAKNSLYLQMNSLRAEDTAVYCAKVSYLSTASSLDYWGQGTLVTVSSASTKGPSV

SEQ ID NO: 29 - protein, 15 aa, synthetic construct
  REGION 1..15: note = synthetic polypeptide
DIQMTQSPSSLSASV

SEQ ID NO: 30 - protein, 15 aa, synthetic construct
  REGION 1..15: note = artificial polypeptide
MTQSPSSLSASVGDR

SEQ ID NO: 31 - protein, 15 aa, synthetic construct
  REGION 1..15: note = synthetic polypeptide
SPSSLSASVGDRVTI

SEQ ID NO: 32 - protein, 14 aa, synthetic construct
  REGION 1..14: note = synthetic polypeptide
SLSASVGDRVTITC

SEQ ID NO: 33 - protein, 15 aa, synthetic construct
  REGION 1..15: note = synthetic polypeptide
ASVGDRVTITCRASQ

(19) United States
(12) Patent Application Publication
(10) Pub. No.: US 2024/0255522 A1
(43) Pub. Date: Aug. 1, 2024

(54) ASSAYS AND REAGENTS FOR CHARACTERIZATION OF MHCII PEPTIDE BINDING

(71) Applicant: GENENTECH, INC., South San Francisco, CA (US)

(72) Inventors: Adel Mahmoud ELSOHLY, South San Francisco, CA (US); Joshua Gregory GOBER, South San Francisco, CA (US); Juan LI, South San Francisco, CA (US)

(21) Appl. No.: 18/589,276

(22) Filed: Feb. 27, 2024

Related U.S. Application Data
(63) Continuation of application No. PCT/US2022/075608, filed on Aug. 29, 2022.
(60) Provisional application No. 63/238,728, filed on Aug. 30, 2021.

(51) Int. Cl. G01N 33/68; G01N 33/58

(52) U.S. Cl. CPC G01N 33/6878; G01N 33/582; G01N 33/6848; G01N 2333/70539

(57) ABSTRACT

Described herein are reagents and methods for high-throughput screening of antigen binding to MHCII alleles.

### CROSS REFERENCE TO RELATED APPLICATION

This application is an international stage application which claims the benefit of priority to U.S. Provisional Application No. 63/238,728, filed Aug. 30, 2021, which is herein incorporated by reference in its entirety.

### SEQUENCE LISTING

The material in the accompanying Sequence Listing is hereby incorporated by reference in its entirety. The accompanying file, named “048893-534001WO_SL_ST26.xml” was created on Aug. 29, 2022 and is 42,603 bytes. The file can be accessed using Microsoft Word on a computer that uses Windows OS.

### BACKGROUND

Major histocompatibility complex II (MHCII) is a group of cell surface proteins, located on the extracellular surfaces of antigen presenting cells (APC) and certain immune cells. MHCII proteins are encoded by the human leukocyte antigen (HLA) locus, by classical alleles HLA-DP, -DQ, and -DR, and non-classical alleles HLA-DM and -DO. MHCII proteins are heterodimers of an alpha chain and a beta chain, which undergo a multi-step maturation process into the final antigen-presenting form. The alpha and beta chains are translated as single-pass membrane proteins in the endoplasmic reticulum, and then associate with the invariant chain (Ii) to form a complex. After cycling between the Golgi, plasma membrane, endosomes, and finally the intraluminal vesicles of multivesicular bodies, the invariant chain is processed by a cathepsin into a smaller peptide, the Class-II associated invariant chain peptide (CLIP), which is bound in the in the peptide-binding groove of the MHCII alpha/beta heterodimer. In subsequent steps, CLIP is exchanged for antigenic peptides via a chaperoned process, and the MHCII/antigenic peptide complex returns to the APC cell surface to present to CD4(+) T-cells.

Antigen presenting cells modulate the adaptive immune response. The adaptive immune response is responsible for: retaining memory of pathogenic attacks, vaccinations, and other non-self antigens, and training helper, or CD4(+) T-cells to regulate various types of assaults in a specific manner. Recently, there is great interest in generating non-self antigens for targeted therapeutics for cancer, novel viral, bacterial, and fungal infections, and certain autoimmune diseases. Indeed, the ability to generate and select for antigens, or neoantigens, in vitro, would accelerate and broaden our abilities to address these disease threats.

### SUMMARY

The instant technology generally relates to reagents and methods for high-throughput screening of peptides to MHCII complexes.

In an aspect, provided herein, is a composition including a test peptide and a major histocompatibility complex class II (MHCII)-ligand complex, the MHCII-ligand complex containing: (i) an MHC molecule containing an alpha chain and a beta chain, and (ii) a ligand associated with the alpha chain and the beta chain.

In an aspect, provided herein, is a major histocompatibility complex class II (MHCII)-ligand complex containing (i) an MHCII molecule containing an alpha chain and a beta chain, and (ii) a ligand associated with the MHCII molecule. The MHCII-ligand complex further contains a ligand with an amino acid sequence selected from: SEQ ID NO.: 1 to SEQ ID NO.: 3.

In an aspect, provided herein, is a method of detecting binding of a major histocompatibility complex class II (MHCII) molecule to a peptide, including: providing a first composition containing a peptide and a MHCII-ligand complex containing (i) the MHCII molecule containing an alpha chain and a beta chain and (ii) a ligand, wherein the ligand is bound to the alpha chain or the beta chain via a cleavable linker; subjecting the first composition to a condition to cause cleavage of the cleavable linker; incubating the first composition for a period of time sufficient to form a second composition, the second composition containing the alpha chain, the beta chain, the ligand, free peptide, and/or a MHCII-peptide complex comprising the MHCII molecule and the peptide non-covalently bound to the MHCII molecule; and determining whether the MHCII molecule is bound to the peptide.

In an aspect, provided herein, is a method for detecting the affinity of a test peptide for a major histocompatibility complex class II (MHCII) molecule, including: providing a first composition containing the test peptide; a tagged MHCII-binding peptide (tagged peptide); and a MHCII-ligand complex containing (i) the MHCII molecule containing an alpha chain and a beta chain and (ii) a ligand, wherein the ligand is bound to the alpha chain or the beta chain via a cleavable linker; subjecting the first composition to a condition to cause cleavage of the cleavable linker; incubating the first composition for a period of time sufficient form a second composition, the second composition containing the alpha chain, the beta chain, the ligand, free test peptide, free tagged peptide, MHCII-tagged peptide complex containing the tagged peptide associated with the MHCII molecule, and/or MHCII-test peptide complex containing the test peptide associated with the MHCII molecule; and determining whether the MHCII molecule is bound to the test peptide.

In an aspect, provided herein, is a method for multiplex epitope mapping for a major histocompatibility complex class II (MHCII) allele, including: providing a first composition comprising a plurality of test peptides, and a plurality of MHCII-ligand complexes, each MHCII-ligand complex containing (i) a MHCII molecule containing an alpha chain and a beta chain and (ii) a ligand, wherein the ligand is bound to the alpha chain or the beta chain via a cleavable linker; subjecting the first composition to a condition to cause cleavage of the cleavable linker; incubating the first composition for a period of time sufficient to form a second composition, the second composition containing the alpha chain, the beta chain, the ligand, free test peptide, and/or a plurality of MHCII-test peptide complexes, each MHCII-test peptide complex comprising the MHCII molecule and a test peptide non-covalently bound thereto; and determining whether one or more of the plurality of test peptides is bound to the MHCII molecule in the second composition.

In an aspect, provided herein, is a method for multiplex epitope mapping for a major histocompatibility complex class II (MHCII) allele, including: providing a first composition containing a plurality of test peptides, and a plurality of MHCII-ligand complexes, each MHCII-ligand complex containing (i) a MHCII molecule containing an alpha chain and a beta chain and (ii) a ligand, wherein the ligand is bound to the alpha chain or the beta chain via a cleavable linker; subjecting the first composition to a condition to cause cleavage of the cleavable linker; incubating the first composition for a period of time sufficient to form a second composition, the second composition containing the alpha chain, the beta chain, the ligand, free test peptide, and/or a plurality of MHCII-test peptide complexes, each MHCII-test peptide complex containing the MHCII molecule and a test peptide non-covalently bound thereto; and determining whether one or more of the plurality of test peptides is bound to the MHCII molecule in the second composition.

In an aspect, provided herein, is a peptide that binds at least one major histocompatibility complex class II (MHCII) molecule, the peptide containing an amino acid sequence selected from: SEQ ID NO.: 1 to SEQ ID NO.: 16.

### BRIEF DESCRIPTION OF THE DRAWINGS

FIG. 1 is a schematic of a peptide exchange assay for MHCII protein complex. An MHCII alpha chain, beta chain, and a covalently-linked ligand (e.g., CLIP ligand) are expressed and purified. The covalently-attached ligand is removed via a peptide backbone cleavage reaction, while in the presence of an excess of test peptide. If test peptide binds MHCII and effectively competes with the CLIP ligand, a MHCII-test peptide complex is formed. The MHCII/test peptide complex is separated by size-exclusion chromatography and the MHCII/test peptide complexes are analyzed via reverse-phase LC-MS.

FIG. 2 is a Coomassie-stained SDS-PAGE gel run showing the expression of MHCII proteins in HEK-293 and CHO cells.

FIGS. 3A and 3B provide an example of a covalently-linked high-affinity CLIP (class-II-associated invariant chain peptide, sequence PVSQMRMATPLLMRP, SEQ ID NO.: 4) enzymatic processing and subsequent exchange reaction with a test peptide (OVA peptide, sequence ISQVHAAHAEINEAGR, SEQ ID NO.: 5) for an MHCII heterodimer. FIG. 3A shows the CLIP peptide is removed by processing by thrombin or TEV protease, where the enzyme cleavage site is located within a linker sequence between the CLIP peptide and N-terminus of the MHCII β-chain. The exchange reaction is initiated by incubating the MHCII/cleaved CLIP peptide complexes overnight in the presence of 20 times the concentration of test peptide (OVA) in acetate buffer at pH 5.2, 37° C. The reaction mixture was separated via size-exclusion chromatography, and analyzed via RP LC-MS. FIG. 3B shows the resulting LC-MS chromatogram from the MHCII/peptide complex size-exclusion fraction indicates the amount of MHCII/OVA peptide (5.2 minutes) vs. MHCII/CLIP peptide (7 minutes).

FIGS. 4A and 4B provide an example of a covalently-linked low-affinity CLIP (class-II-associated invariant chain peptide, sequence PVSQARMATGALARP, SEQ ID NO.: 6) enzymatic processing and subsequent exchange reaction with a test peptide (OVA peptide, sequence ISQVHAAHAEINEAGR, SEQ ID NO.: 5) for an MHCII heterodimer. FIG. 4A shows the CLIP peptide is removed by processing by thrombin or TEV protease, where the enzyme cleavage site is located within a linker sequence between the CLIP peptide and N-terminus of the MHCII β-chain. The exchange reaction is initiated by incubating the MHCII/cleaved CLIP peptide complexes overnight in the presence of 20 times the concentration of test peptide (OVA) in acetate buffer at pH 5.2, 37° C. The reaction mixture was separated via size-exclusion chromatography, and analyzed via RP LC-MS. FIG. 4B shows the resulting LC-MS chromatogram from the MHCII/peptide complex size-exclusion fraction indicates the amount of MHCII/OVA peptide (5.2 minutes) vs. MHCII/CLIP peptide (˜6.8 minutes).

FIGS. 5A and 5B show an SEC chromatogram from the first dimension of the 2D LC-MS (FIG. 5A), and the extracted ion chromatogram from the LC-MS second dimension detection (FIG. 5B), showing the corresponding counts vs. acquisition time windows showing the differences in three separate exchange assay outcomes for HLA-DRB1*01:01 complex with three different CLIP peptide sequences, and the same test peptide sequence (pp65, LPLKMLNIPSINVH, SEQ ID NO.: 7).

FIGS. 6A and 6B show an LC-MS chromatogram (FIG. 6A) and the corresponding counts vs. acquisition time windows (FIG. 6B) showing the differences in three separate exchange assay outcomes for HLA-DRB1*03:01 complex with three different CLIP peptide sequences, and the same test peptide sequence (SP41, HTYTIDWTKDAVTWS, SEQ ID NO.: 8).

FIGS. 7A and 7B show an LC-MS chromatogram (FIG. 7A) and the corresponding counts vs. acquisition time windows (FIG. 7B) showing the differences in three separate exchange assay outcomes for HLA-DRB1*04:01 complex with three different CLIP peptide sequences, and the same test peptide sequence (HA, PKYVKQNTLKLAT, SEQ ID NO.: 9).

FIGS. 8A and 8B show an LC-MS chromatogram (FIG. 8A) and the corresponding counts vs. acquisition time windows (FIG. 8B) showing the differences in three separate exchange assay outcomes for HLA-DRB1*07:01 complex with three different CLIP peptide sequences, and the same test peptide sequence (pp65, EPDVYYTSAFVFPTK, SEQ ID NO.: 10).

FIGS. 9A and 9B show an LC-MS chromatogram (FIG. 9A) and the corresponding counts vs. acquisition time windows (FIG. 9B) showing the differences in three separate exchange assay outcomes for HLA-DRB1*07:01 complex with three different CLIP peptide sequences, and the same test peptide sequence (HA, PKYVKQNTLKLAT, SEQ ID NO.: 9).

FIG. 10 is a table of CLIP variants (or wild type CLIP) identified for HLA-DR alleles.

FIGS. 11A-11C show a table of test peptides for MHCII binding (FIG. 11A), a cartoon depiction of a complex, including a fluorescent avidin tetramer of biotinylated MHCII molecules (FIG. 11B), with a test peptide associated, to use for screening murine T-cells, and the subsequent flow cytometry experiment+/−CD44 T-cell screen. FIG. 11C is an interferon gamma ELISPOT assay that shows activation of T cells to the shorter mutated peptide identified from peptide exchange as the only binder. The short peptide identified gives a comparable ELISPOT response as the long, synthetic peptide (M112_7788-5).

FIG. 12 shows multiplexed analysis of peptide binding of a 150 peptide library from Humira and Remicade variable domains in a single exchange.

FIG. 13 is a schematic of a competitive peptide exchange assay. A low affinity containing peptide is covalently attached to an MHCII molecule, via a cleavable linker. The linker is cleaved in the presence of a pool of labeled, high affinity peptide and a test peptide(s), and the low affinity peptide is exchanged. The three outcomes illustrated are: MHCII molecule binds the high-affinity peptide, and is detected, the MHCII molecule binds the test peptide(s), and is not detected, and/or the MHCII complex becomes unable to bind a peptide, and is not detected.

FIGS. 14A-14C show an example workflow for screening MHCII and peptide complexes via ELISA. The cartoon schematic in FIG. 14A illustrates a competitive peptide exchange time-course for MHCII molecules and a pool containing a labeled high affinity peptide (biotinylated, for example), and a test peptide(s) at various concentration ranges. The peptide exchange reaction mix is incubated for a certain time and at a certain pH (for example, 60-72 hours at pH 5), and then applied to an ELISA plate coated with an anti-MHC antibody. The loaded ELISA plate is developed by adding a developing reagent (streptavidin-horse radish peroxidase, for example). The resulting signal is plotted vs. concentration, and resulting data points are fit to a binding model, and a binding affinity is calculated for the test peptide. FIG. 14B is a plot showing the binding curves and affinity ranking for eight high affinity peptides for MHCII. FIG. 14C is a table containing the IC₅₀ values (binding affinity) of each peptide for MHCII, along with the R² value of the model fit.

FIGS. 15A-15G show normalized signal vs. log concentration plots of HLA-DRB1 alleles/low affinity ligand peptide complexes, a labeled high affinity peptide (biotinylated huCLIP, 50 nM) and a varied concentration range of eight different test peptides. All results are measured by ELISA. FIG. 15A is the log plot of the competitive binding for DRB1*01:01 and peptides mCLIP, LCMV RNAP, LASV RNAP, VETF (275-289), LCMV NP (201-215), VV HP (23-37), MOG (35-55), and OVA (323-339). FIG. 15B is the log plot of the competitive binding for DRB1*08:01 and peptides mCLIP, LCMV RNAP, LASV RNAP, VETF (275-289), LCMV NP (201-215), VV HP (23-37), MOG (35-55), and OVA (323-339). FIG. 15C is the log plot of the competitive binding for DRB1*15:01 and peptides mCLIP, LCMV RNAP, LASV RNAP, VETF (275-289), LCMV NP (201-215), VV HP (23-37), MOG (35-55), and OVA (323-339).

FIG. 15D is the log plot of the competitive binding for DRB1*04:01 and peptides mCLIP, LCMV RNAP, LASV RNAP, VETF (275-289), LCMV NP (201-215), VV HP (23-37), MOG (35-55), and OVA (323-339). FIG. 15E is the log plot of the competitive binding for DRB1*09:01 and peptides mCLIP, LCMV RNAP, LASV RNAP, VETF (275-289), LCMV NP (201-215), VV HP (23-37), MOG (35-55), and OVA (323-339). FIG. 15F is the log plot of the competitive binding for DRB1*11:01 and peptides mCLIP, LCMV RNAP, LASV RNAP, VETF (275-289), LCMV NP (201-215), VV HP (23-37), MOG (35-55), and OVA (323-339). FIG. 15G Reference pp65-biotinylated DRB1*01:01.

FIGS. 16A and 16B show the saturation binding curves of normalized signal vs. peptide concentration for eight different HLA DRB1 allele samples/low affinity ligand peptide complexes (at 100 nM), and one high affinity peptide (biotinylated huCLIP). The concentration range for the high affinity CLIP peptide is 40,000 nM-2.4 nM. The peptide exchange time course was run over 70 hours, at 37° C., at pH 5 (FIG. 16A). FIG. 16B is a table of the binding affinities values for eight different HLA DRB1 allele samples and the high affinity peptide (biotinylated huCLIP). The data were fit to a Specific Binding with Hill slope model, where Bₘₐₓ is the maximum specific binding, K_{d} is the half-maximum ligand binding at equilibrium, h is the Hill slope, and R squared value is how well the model fits the data.

FIGS. 17A-17C are a determination of optimal biotinylated-huCLIP (huCLIP-bio) peptide concentration for an allele-specific probe in a binding competition assay vs. mCLIP as a test peptide. A peptide exchange assay was performed for six DRB1 alleles and a reference allele (pp65-DRB1*01:01). FIG. 17A shows the plot of relative signal vs. log of mCLIP concentration range (40,000-0.38 nM) for 50 nM huCLIP-bio, and FIG. 17B shows the plot of relative signal vs. log of mCLIP concentration range (40,000-0.38 nM) for 25 nM huCLIP-bio. FIG. 17C is a table of the calculated K_{d} values.

FIGS. 18A-18F show plots of peptide exchange assays performed with either 50 nM or 25 nM huCLIP-bio as the high affinity peptide for DRB1*01:01, DRB1*08:01, and DRB1*11:01. FIG. 18A and FIG. 18D are the plots for 50 nM and 25 nM hCLIP-bio used to compete against mCLIP, LCMV RNAP, LASV RNAP, VETF (275-289), LCMV NP (201-215), VV HP (23-37), MOG (35-55), and OVA (323-339) test peptides for DRB1*08:01 binding. FIG. 18B and FIG. 18E are the plots for 50 nM and 25 nM hCLIP-bio used to compete against mCLIP, LCMV RNAP, LASV RNAP, VETF (275-289), LCMV NP (201-215), VV HP (23-37), MOG (35-55), and OVA (323-339) test peptides for DRB1*11:01 binding. FIG. 18C and FIG. 18F are the plots for 50 nM and 25 nM hCLIP-bio used to compete against mCLIP, LCMV RNAP, LASV RNAP, VETF (275-289), LCMV NP (201-215), VV HP (23-37), MOG (35-55), and OVA (323-339) test peptides for DRB1*11:01 binding.

FIGS. 19A-19D show plots of peptide exchange assays performed with either 50 nM or 25 nM huCLIP-bio for DRB1*04:01 and DRB1*15:01. FIG. 19A and FIG. 19B are the plots for 50 nM hCLIP-bio used to compete against mCLIP, LCMV RNAP, LASV RNAP, VETF (275-289), LCMV NP (201-215), VV HP (23-37), MOG (35-55), and OVA (323-339) test peptides for DRB1*04:01 and DRB1*15:01 binding, respectively. FIG. 19C and FIG. 19D are the plots for 25 nM hCLIP-bio used to compete against mCLIP, LCMV RNAP, LASV RNAP, VETF (275-289), LCMV NP (201-215), VV HP (23-37), MOG (35-55), and OVA (323-339) test peptides for DRB1*04:01 and DRB1*15:01 binding, respectively.

FIGS. 20A and 20B show plots of peptide exchange assays performed with either 50 nM or 25 nM huCLIP-bio for DRB1*09:01. The plots for 50 nM (FIG. 20A) and 25 nM (FIG. 20B) hCLIP-bio used to compete against mCLIP, LCMV RNAP, LASV RNAP, VETF (275-289), LCMV NP (201-215), VV HP (23-37), MOG (35-55), and OVA (323-339) test peptides for DRB1*09:01 binding.

FIG. 21 is a workflow diagram example for the peptide exchange assay.

FIG. 22 is a table of the test peptide sequences.

FIGS. 23A-23E show plots of peptide exchange assays performed with either 50 nM or 25 nM huCLIP-bio (FIGS. 23A and 23B), an overlay of SEC chromatograms for the completed peptide exchange reactions, displaying the MHCII/peptide complex peak at 2.5 minutes (FIG. 23C), an overlay of the counts vs. acquisition time for each SEC peak, indicating the amount of test peptide exchanged for the low affinity CLIP (FIG. 23D), and a table of test peptides sequences, and their resulting exchange profile for each test peptide as compared to the low affinity CLIP peptide, for MHCII HLA allele DRB1*01:01 (FIG. 23E). The concentration of MHCII was 16 μM and the concentration of test peptide was 400 μM. The test peptide sequences are as indicated in FIG. 22.

FIGS. 24A-24E show plots of peptide exchange assays performed with either 50 nM or 25 nM huCLIP-bio (FIGS. 24A and 24B), an overlay of SEC chromatograms for the completed peptide exchange reactions, displaying the MHCII/peptide complex peak at 2.5 minutes (FIG. 24C), an overlay of the counts vs. acquisition time for each SEC peak, indicating the amount of test peptide exchanged for the low affinity CLIP (FIG. 24D), and a table of test peptides sequences (FIG. 24E), and their resulting exchange profile for each test peptide as compared to the low affinity CLIP peptide, for MHCII HLA allele DRB1*04:01. The concentration of MHCII was 16 μM and the concentration of test peptide was 400 μM. The test peptide sequences are as indicated in FIG. 22.

FIGS. 25A-25E show plots of peptide exchange assays performed with either 50 nM or 25 nM huCLIP-bio (FIGS. 25A and 25B), an overlay of SEC chromatograms for the completed peptide exchange reactions, displaying the MHCII/peptide complex peak at 2.5 minutes (FIG. 25C), an overlay of the counts vs. acquisition time for each SEC peak, indicating the amount of test peptide exchanged for the low affinity CLIP (FIG. 25D), and a table of test peptides sequences (FIG. 25E), and their resulting exchange profile for each test peptide as compared to the low affinity CLIP peptide, for MHCII HLA allele DRB1*08:01. The concentration of MHCII was 16 μM and the concentration of test peptide was 400 μM. The test peptide sequences are as indicated in FIG. 22.

FIGS. 26A-26D show plots of peptide exchange assays performed with either 50 nM or 25 nM huCLIP-bio (FIGS. 26A and 26B), an overlay of SEC chromatograms for the completed peptide exchange reactions, displaying the MHCII/peptide complex peak at 2.5 minutes (FIG. 26C), and an overlay of the counts vs. acquisition time for each SEC peak, indicating the amount of test peptide exchanged for the low affinity CLIP (FIG. 26D). The concentration of MHCII was 16 μM and the concentration of test peptide was 400 μM. The test peptide sequences are as indicated in FIG. 22.

FIGS. 27A-27E show plots of peptide exchange assays performed with either 50 nM or 25 nM huCLIP-bio (FIGS. 27A and 27B), an overlay of SEC chromatograms for the completed peptide exchange reactions, displaying the MHCII/peptide complex peak at 2.5 minutes (FIG. 27C), an overlay of the counts vs. acquisition time for each SEC peak, indicating the amount of test peptide exchanged for the low affinity CLIP (FIG. 27D), and a table of test peptides sequences (FIG. 27E), and their resulting exchange profile for each test peptide as compared to the low affinity CLIP peptide, for MHCII HLA allele DRB1*11:01. The concentration of MHCII was 16 μM and the concentration of test peptide was 400 μM. The test peptide sequences are as indicated in FIG. 22.

FIGS. 28A-28B show plots of peptide exchange assays performed with either 50 nM or 25 nM huCLIP-bio (FIGS. 28A and 28B), an overlay of SEC chromatograms for the completed peptide exchange reactions, displaying the MHCII/peptide complex peak at 2.5 minutes (FIG. 28C), an overlay of the counts vs. acquisition time for each SEC peak, indicating the amount of test peptide exchanged for the low affinity CLIP (FIG. 28D), and a table of test peptides sequences (FIG. 28E), and their resulting exchange profile for each test peptide as compared to the low affinity CLIP peptide, for MHCII HLA allele DRB1*15:01. The concentration of MHCII was 16 μM and the concentration of test peptide was 400 μM. The test peptide sequences are as indicated in FIG. 22.

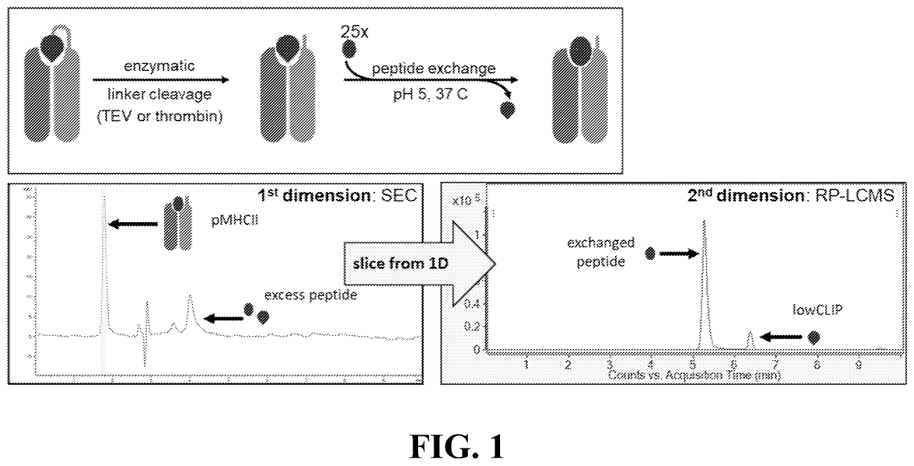

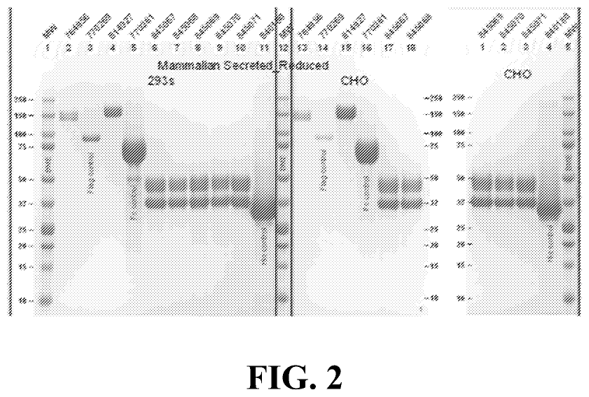

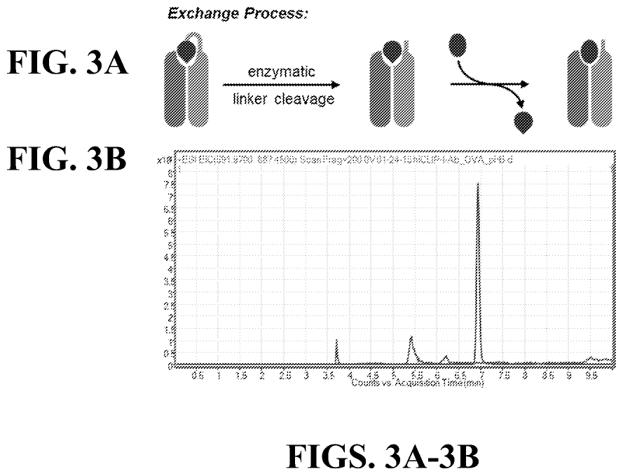

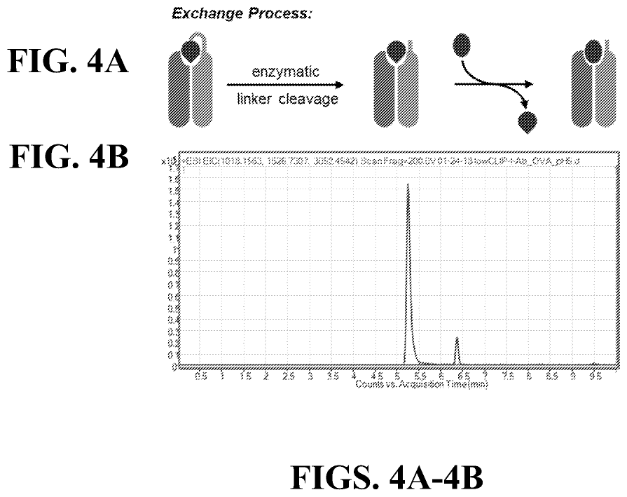

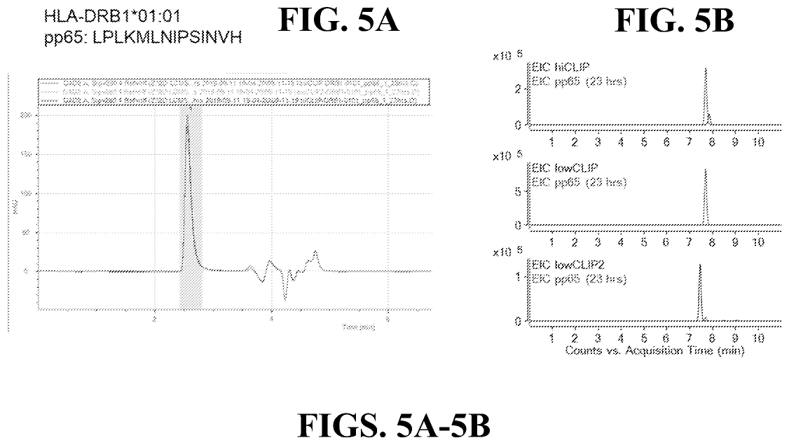

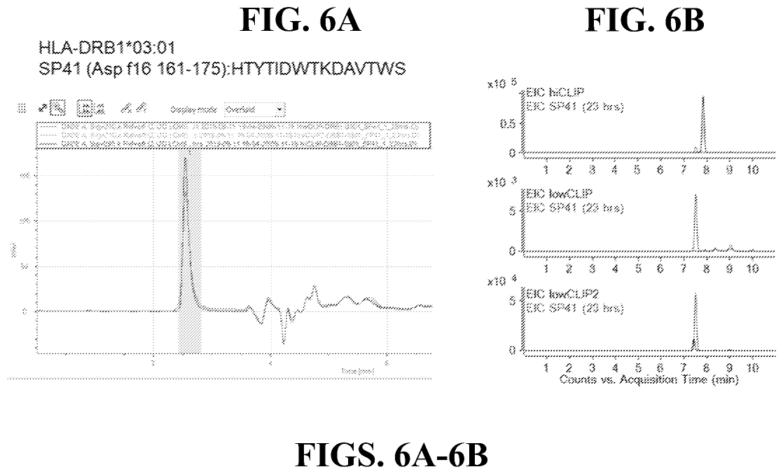

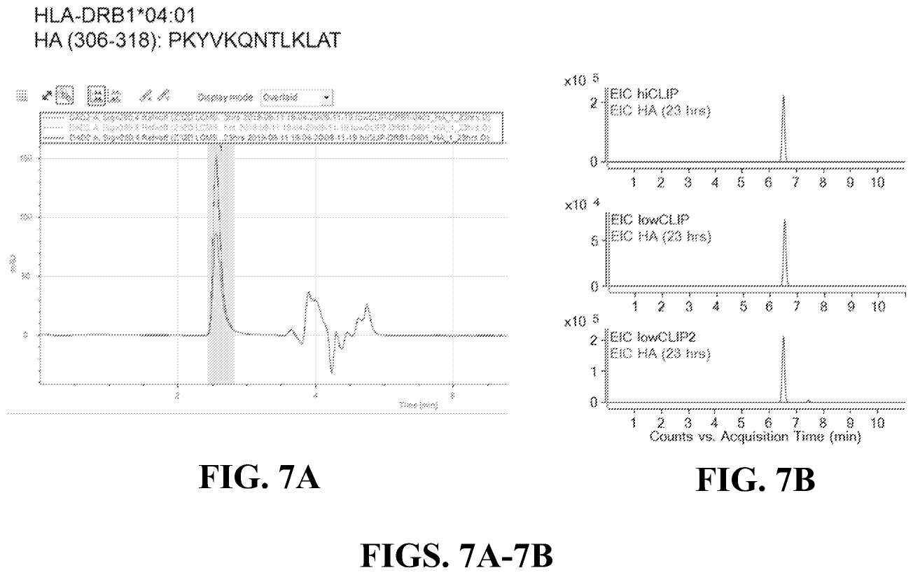

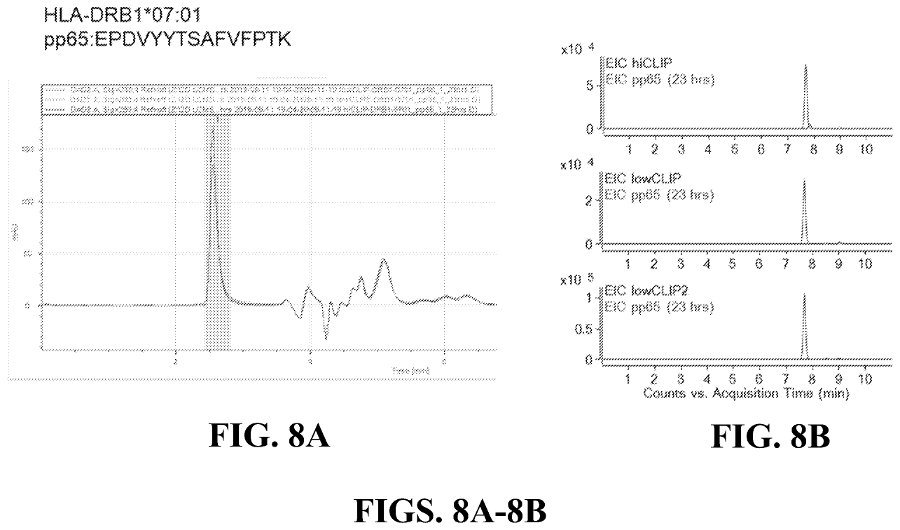

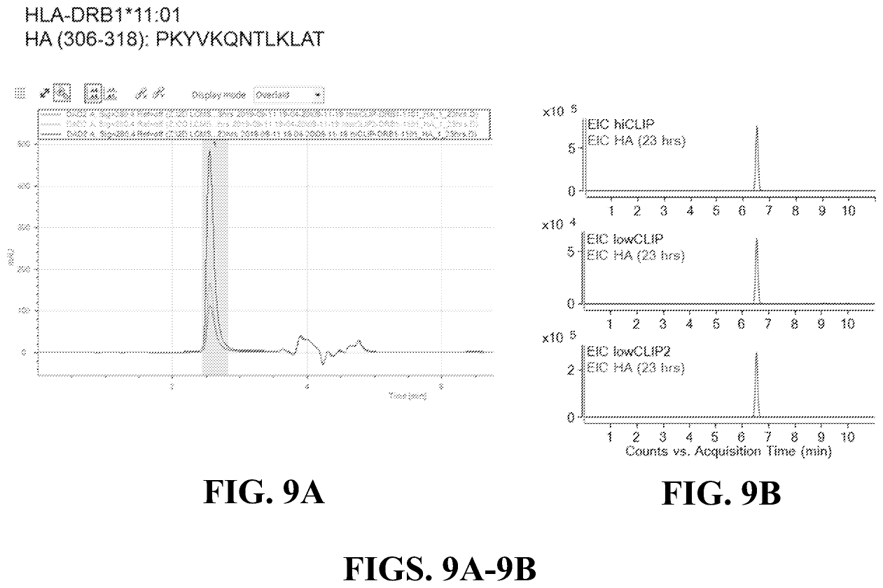

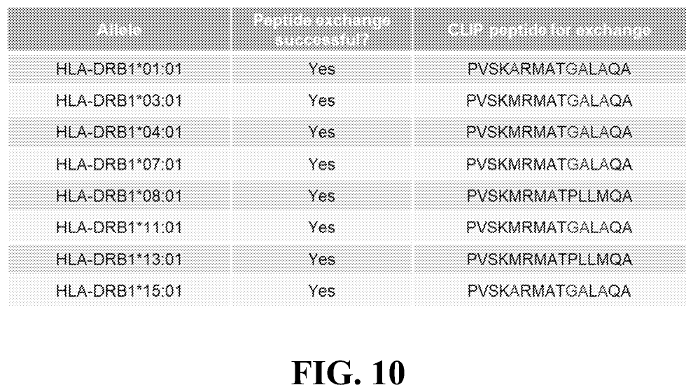

### DETAILED DESCRIPTION

After reading this description it will become apparent to one skilled in the art how to implement the present disclosure in various alternative embodiments and alternative applications. However, all the various embodiments of the present invention will not be described herein. It will be understood that the embodiments presented here are presented by way of an example only, and not limitation. As such, this detailed description of various alternative embodiments should not be construed to limit the scope or breadth of the present disclosure as set forth herein.

Before the present technology is disclosed and described, it is to be understood that the aspects described below are not limited to specific compositions, methods of preparing such compositions, or uses thereof as such may, of course, vary. It is also to be understood that the terminology used herein is for the purpose of describing particular aspects only and is not intended to be limiting.

The instant application provides compositions and methods for assaying MHCII peptides for use in a wide range of immunotherapies.

In an aspect, provided herein, is a major histocompatibility complex class II (MHCII) protein complex, including an alpha chain, a beta chain, and a ligand, where the ligand is associated with the alpha chain and beta chain.

In an aspect, provided herein, is a major histocompatibility complex class II (MHCII)-ligand complex, including an MHCII alpha chain and an MHCII beta chain, and a ligand associated with the MHCII molecule, where the ligand is a test peptide.

In an aspect, provided herein, is a method of detecting binding of a major histocompatibility complex class II (MHCII) molecule to a peptide, including: providing a first composition including a peptide and a MHCII-ligand complex, where the MHCII-ligand complex includes an alpha chain and a beta chain and a ligand, wherein the ligand is bound to the alpha chain or the beta chain via a cleavable linker; subjecting the first composition to a condition to cause cleavage of the cleavable linker; incubating the first composition for a period of time sufficient to form a second composition, the second composition including the alpha chain, the beta chain, the ligand, free peptide, and/or a MHCII-peptide complex including the MHCII molecule and the peptide non-covalently bound to the MHCII molecule. In embodiments, the method includes determining whether the MHCII molecule is bound to the peptide.

In an aspect, provided herein, is a method for detecting the affinity of a test peptide for a major histocompatibility complex class II (MHCII) molecule, including: providing a first composition including the test peptide; a tagged MHCII-binding peptide (tagged peptide); and a MHCII-ligand complex including (i) the MHCII molecule including an alpha chain and a beta chain and (ii) a ligand, wherein the ligand is bound to the alpha chain or the beta chain via a cleavable linker; subjecting the first composition to a condition to cause cleavage of the cleavable linker; and incubating the first composition for a period of time sufficient form a second composition, the second composition including the alpha chain, the beta chain, the ligand, free test peptide, free tagged peptide, MHCII-tagged peptide complex including the tagged peptide associated with the MHCII molecule, and/or MHCII-test peptide complex including the test peptide associated with the MHCII molecule.

In embodiments, the method further includes: determining whether the MHCII molecule is bound to the test peptide.

The detailed description divided into various sections only for the reader's convenience and disclosure found in any section may be combined with that in another section. Titles or subtitles may be used in the specification for the convenience of a reader, which are not intended to influence the scope of the present disclosure.

### Definitions

Unless defined otherwise, all technical and scientific terms used herein have the same meaning as commonly understood by one of ordinary skill in the art to which this disclosure belongs. In this specification and in the claims that follow, reference will be made to a number of terms that shall be defined to have the following meanings:

The terminology used herein is for the purpose of describing particular embodiments only and is not intended to be limiting. As used herein, the singular forms “a”, “an” and “the” are intended to include the plural forms as well, unless the context clearly indicates otherwise.

“Optional” or “optionally” means that the subsequently described event or circumstance can or cannot occur, and that the description includes instances where the event or circumstance occurs and instances where it does not.

The term “about” when used before a numerical designation, e.g., temperature, time, amount, concentration, and such other, including a range, indicates approximations which may vary by (+) or (−) 10%, 5%, 1%, or any subrange or subvalue there between. Preferably, the term “about” when used with regard to an amount means that the amount may vary by +/−10%.

“Comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this disclosure.

As used herein, the term “cancer” refers to all types of cancer, neoplasm or malignant tumors found in mammals (e.g. humans), including leukemias, lymphomas, carcinomas and sarcomas. Exemplary cancers that may be treated with a compound or method provided herein include brain cancer, glioma, glioblastoma, neuroblastoma, prostate cancer, colorectal cancer, pancreatic cancer, Medulloblastoma, melanoma, cervical cancer, gastric cancer, ovarian cancer, lung cancer, cancer of the head, Hodgkin's Disease, and Non-Hodgkin's Lymphomas. Exemplary cancers that may be treated with a compound or method provided herein include cancer of the thyroid, endocrine system, brain, breast, cervix, colon, head & neck, liver, kidney, lung, ovary, pancreas, rectum, stomach, and uterus. Additional examples include, thyroid carcinoma, cholangiocarcinoma, pancreatic adenocarcinoma, skin cutaneous melanoma, colon adenocarcinoma, rectum adenocarcinoma, stomach adenocarcinoma, esophageal carcinoma, head and neck squamous cell carcinoma, breast invasive carcinoma, lung adenocarcinoma, lung squamous cell carcinoma, non-small cell lung carcinoma, mesothelioma, multiple myeloma, neuroblastoma, glioma, glioblastoma multiforme, ovarian cancer, rhabdomyosarcoma, primary thrombocytosis, primary macroglobulinemia, primary brain tumors, malignant pancreatic insulanoma, malignant carcinoid, urinary bladder cancer, premalignant skin lesions, testicular cancer, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

“Selective” or “selectivity” or the like of a compound refers to the compound's ability to discriminate between molecular targets (e.g. a compound having selectivity toward HMT SUV39H1 and/or HMT G9a).

“Specific”, “specifically”, “specificity”, or the like of a compound refers to the compound's ability to cause a particular action, such as inhibition, to a particular molecular target with minimal or no action to other proteins in the cell (e.g. a compound having specificity towards HMT SUV39H1 and/or HMT G9a displays inhibition of the activity of those HMTs whereas the same compound displays little-to-no inhibition of other HMTs such as DOT1, EZH1, EZH2, GLP, MLL1, MLL2, MLL3, MLL4, NSD2, SET1b, SET7/9, SET8, SETMAR, SMYD2, SUV39H2).

The terms “immune response” and the like refer, in the usual and customary sense, to a response by an organism that protects against disease. The response can be mounted by the innate immune system or by the adaptive immune system, as well known in the art.

The terms “modulating immune response” and the like refer to a change in the immune response of a subject as a consequence of administration of an agent, e.g., a compound as disclosed herein, including embodiments thereof. Accordingly, an immune response can be activated or deactivated as a consequence of administration of an agent, e.g., a compound as disclosed herein, including embodiments thereof.

“B Cells” or “B lymphocytes” refer to their standard use in the art. B cells are lymphocytes, a type of white blood cell (leukocyte), that develops into a plasma cell (a “mature B cell”), which produces antibodies. An “immature B cell” is a cell that can develop into a mature B cell. Generally, pro B cells undergo immunoglobulin heavy chain rearrangement to become pro B pre B cells, and further undergo immunoglobulin light chain rearrangement to become an immature B cells. Immature B cells include T1 and T2 B cells.

“T cells” or “T lymphocytes” as used herein are a type of lymphocyte (a subtype of white blood cell) that plays a central role in cell-mediated immunity. They can be distinguished from other lymphocytes, such as B cells and natural killer cells, by the presence of a T-cell receptor on the cell surface. T cells include, for example, natural killer T (NKT) cells, cytotoxic T lymphocytes (CTLs), regulatory T (Treg) cells, and T helper cells. Different types of T cells can be distinguished by use of T cell detection agents.

A “memory T cell” is a T cell that has previously encountered and responded to its cognate antigen during prior infection, encounter with cancer or previous vaccination. At a second encounter with its cognate antigen memory T cells can reproduce (divide) to mount a faster and stronger immune response than the first time the immune system responded to the pathogen.

A “regulatory T cell” or “suppressor T cell” is a lymphocyte which modulates the immune system, maintains tolerance to self-antigens, and prevents autoimmune disease.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues, wherein the polymer may be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

The term “amino acid side chain” refers to the functional substituent contained on amino acids. For example, an amino acid side chain may be the side chain of a naturally occurring amino acid. Naturally occurring amino acids are those encoded by the genetic code (e.g., alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine), as well as those amino acids that are later modified, e.g., hydroxyproline, γ-carboxyglutamate, and O-phosphoserine. In embodiments, the amino acid side chain may be a non-natural amino acid side chain.

The term “non-natural amino acid side chain” refers to the functional substituent of compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium, allylalanine, 2-aminoisobutryric acid. Non-natural amino acids are non-proteinogenic amino acids that either occur naturally or are chemically synthesized. Such analogs have modified R groups (e.g., norleucine) or modified peptide backbones, but retain the same basic chemical structure as a naturally occurring amino acid.

The term “MHCII” or “major histocompatibility complex class II” or “major histocompatibility complex II” as provided herein includes any of the recombinant or naturally-occurring forms of the major histocompatibility complex II (MHCII) or variants or homologs thereof that maintain MHCII activity (e.g. within at least 50%, 80%, 90%, 95%, 96%, 97%, 98%, 99% or 100% activity compared to MHCII). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring MHCII polypeptide. In embodiments, MHCII is a heterodimer of two non-covalently bound proteins, an alpha (α) chain and a beta chain (β), homolog or functional fragment thereof. In embodiments, MHCII includes a peptide ligand.

The term “HLA” or “human leukocyte antigen” refers to a group of proteins encoded by the MHC gene complex. Specifically, but not limited to, the MHCII gene complex encodes for the HLA-DP, HLA-DM, HLA-DOA, HLA-DOB, HLA-DQ and HLA-DR group of proteins.

The term “ligand” refers to a molecule that forms a complex with a biomolecule to serve a biological function. Binding can take place between, and not limited to, proteins, peptides, RNA, DNA, nucleic acids, nucleic acid derivatives, non-natural nucleic acids, amino acids, amino acid derivatives, non-natural amino acids, carbohydrates, monosaccharides, disaccharides, oligosaccharides, oligonucleotides, metals, metal complexes, drugs, lipids, fatty acids, metabolites, inorganic molecules, organic molecules, biopolymers, and polymers. Ligand complexes can be formed via ionic bonding, covalent bonding, Van der Waals interactions, and/or hydrogen bonding. Ligands for MHCII are generally peptides.

The term “Class II-associated invariant chain peptide” and “CLIP” refer to part of the invariant chain (Ii) protein sequence that binds in the MHCII peptide-binding groove during the affinity maturation process.

The terms “bind” and “bound” as used herein is used in accordance with its plain and ordinary meaning and refers to the association between atoms or molecules. The association can be direct or indirect. For example, bound atoms or molecules may be direct, e.g., by covalent bond or linker (e.g. a first linker or second linker), or indirect, e.g., by non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like).

The term “antibody” refers to a polypeptide encoded by an immunoglobulin gene or functional fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies can be selected to obtain only a subset of antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998) for a description of immunoassay formats and conditions that can be used to determine specific immunoreactivity).

The term “denature” refers to a process where the three-dimensional structure of a protein, polypeptide, DNA, RNA, or other biopolymer is disrupted by chemical or mechanical means, or by heating or cooling.

The term “peptide linker” refers to an amino acid sequence that links one peptide to another. The peptide linker may be a cleavable linker. The cleavable linker may be any cleavable linker. For example, and without limitation, a cleavable linker may be a UV-cleavable linker, an enzyme-cleavable linker, a pH-dependent linker, a salt-dependent linker, and the like. Preferably, the peptide linker or cleavable linker linking a peptide to a MHCII subunit does not result in the peptide binding more strongly to MHCII than in the absence of the linker. A cleavable linker may made of any suitable molecule(s), and are not limited to peptide linkers.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### MHCII Compositions

In an aspect, provided herein, is a major histocompatibility complex class II (MHCII)/ligand complex including a MHCII molecule, which includes an alpha chain, a beta chain, and a ligand, wherein the ligand is a peptide associated with the alpha chain and the beta chain.

In embodiments, the MHCII/ligand complex contains an alpha chain and a beta chain. In embodiments, the alpha chain and beta chain are associated in a heterodimer. In embodiments, the alpha chain contains a first dimerization tag. In embodiments, the beta chain contains a second dimerization tag. In embodiments, the alpha chain containing a first dimerization tag and the beta chain containing a second dimerization tag are associated.

In embodiments, the MHCII/ligand complex contains an alpha chain and a beta chain that are more stable as a heterodimer. In embodiments, the alpha chain containing a first dimerization tag and the beta chain containing a second dimerization tag are associated in a stable complex. In embodiments, the alpha chain containing a first dimerization tag and the beta chain containing a second dimerization tag are associated in a more stable complex than a complex formed by the same alpha chain and beta chain sequences without the first and second dimerization tags.

In embodiments, the MHCII/ligand complex contains an alpha chain containing a first dimerization tag and the beta chain containing a second dimerization tag, wherein the dimerization tag is selected from: a leucine zipper, Fos/Jun, coiled coil heterodimerization tags, knob-in-hole or other heterodimeric Fcs, or an iDimerize pair.

In embodiments, the MHCII/ligand complex contains an alpha chain and a beta chain, and a ligand. In embodiments, the MHCII/ligand complex contains an alpha chain and a beta chain, and a ligand, wherein the ligand is a peptide. In embodiments, the peptide ligand is covalently attached to the alpha chain. In embodiments, the peptide ligand is covalently attached to the alpha chain via a peptide linker sequence. In embodiments, the peptide ligand is covalently attached to the beta chain. In embodiments, the peptide ligand is covalently attached to the beta chain via a peptide linker sequence. In embodiments, the peptide linker sequence contains an enzyme cleavage site. In embodiments, the enzyme cleavage site is selected from: thrombin, enterokinase, Factor Xa, small ubiquitin-like modifier (SUMO) protease, tobacco etch-virus (TEV) protease, PreScission™ protease, rhinovirus 3C protease, Carboxypeptidase A, Carboxypeptidase B, dipeptidyl aminopeptidase (DAP), tobacco vein mottling virus protease (TVMV), and any variants thereof. In some embodiments, the peptide is fused to the N-terminus of the alpha chain with a flexible peptide linker. In some embodiments, the linker contains the thrombin recognition sequence GGGGSLVPRGSGGGGS (SEQ ID NO.: 17). In some embodiments, the linker contains the TEV protease recognition sequence GGGGSENLYFQGGGGGS (SEQ ID NO.: 18).

In embodiments, the MHCII/ligand complex contains an alpha chain and a beta chain, and a ligand, wherein the ligand is a peptide, wherein the ligand peptide is covalently attached to the alpha chain or beta chain via a peptide linker sequence. In embodiments, the peptide linker sequence contains a non-natural amino acid. In embodiments, the non-natural amino acid is UV-cleavable. In some embodiments, the non-natural amino acid is selected from 2-nitrophenylglycine (NPG), expanded o-nitrobenzyl linker, o-nitrobenzylcaged phenol, o-nitrobenzyl caged thiol, 32 nitroveratryloxycarbonyl (NVOC) caged aniline, o-nitrobenzyl caged selenides, bis-azobenzene, coumarin, cinnamyl, spiropyran, 2-nitrophenylalanine (2-nF), and 3-amino-3-(2-nitrophenyl)propionic acid (ANP) amino acid analogs. In some embodiments, the non-natural amino acid is 3-amino-3-(2-nitrophenyl)propionic acid (ANP).

In embodiments, the MHCII complex contains an alpha chain and a beta chain, and a tagged peptide. In embodiments, the tagged peptide selectively recognizes and associates with the MHCII complex without a ligand. In embodiments, the tagged peptide associates with an additional molecule to form a detectable complex.

In embodiments, the MHCII/ligand complex contains an alpha chain and a beta chain, a ligand, and a tagged peptide. In embodiments, the tagged peptide selectively recognizes and associates with a MHCII complex/ligand complex. In embodiments, the tagged peptide associates with an additional molecule to form a detectable complex.

In embodiments, the tagged peptide is capable of binding a molecule to create a detectable label. In embodiments, the tagged peptide contains a fluorescent compound or a bioluminescent compound. In embodiments, the tagged peptide is tagged with a multivalent platform. In embodiments, the tagged peptide is tagged with biotin, and the additional molecule is streptavidin. In embodiments, the tagged peptide is tagged with subunits of a tetramer assembly.

In embodiments, the MHCII/ligand complex contains an alpha chain and a beta chain, a ligand, and an antibody. In embodiments, the antibody binds to MHCII molecule. In embodiments, the antibody is immobilized on a surface.

In embodiments, the MHCII/ligand complex has an affinity for the peptide ligand. In embodiments, the peptide ligand contains a CLIP sequence. In embodiments, the peptide ligand satisfies one or more of the following criteria:
a) produced at usable quantities during expression with an MHCII chain;
b) is detected at high levels in 2D LCMS (after cleavage of linker); and/or
c) has a peptide exchange yield of about 20% to 100% in an exchange reaction with a peptide known to have high affinity for the allele.

In embodiments, the peptide ligand satisfies two or more of the criteria. In embodiments, the peptide ligand satisfies all three of the criteria.

In embodiments, the usable quantity of MHCII/ligand complex expressed is greater than 1 mg/L. In embodiments, the usable quantity of MHCII/ligand complex expressed is between about 1 mg/L and about 300 mg/L. In embodiments, the usable quantity of MHCII/ligand complex expressed is between about 1 mg/L and about 250 mg/L. In embodiments, the usable quantity of MHCII/ligand complex expressed is between about 1 mg/L and about 200 mg/L. In embodiments, the usable quantity of MHCII/ligand complex expressed is between about 1 mg/L and about 190 mg/L. In embodiments, the usable quantity of MHCII/ligand complex expressed is between about 1 mg/L and about 180 mg/L. In embodiments, the usable quantity of MHCII/ligand complex expressed is between about 1 mg/L and about 170 mg/L. In embodiments, the usable quantity of MHCII/ligand complex expressed is between about 1 mg/L and about 160 mg/L. In embodiments, the usable quantity of MHCII/ligand complex expressed is between about 1 mg/L and about 150 mg/L. In embodiments, the usable quantity of MHCII/ligand complex expressed is between about 1 mg/L and about 140 mg/L. In embodiments, the usable quantity of MHCII/ligand complex expressed is between about 1 mg/L and about 130 mg/L. In embodiments, the usable quantity of MHCII/ligand complex expressed is between about 1 mg/L and about 120 mg/L. In embodiments, the usable quantity of MHCII/ligand complex expressed is between about 1 mg/L and about 110 mg/L. In embodiments, the usable quantity of MHCII/ligand complex expressed is between about 1 mg/L and about 100 mg/L. In embodiments, the usable quantity of MHCII/ligand complex expressed is between about 1 mg/L and about 90 mg/L. In embodiments, the usable quantity of MHCII/ligand complex expressed is between about 1 mg/L and about 80 mg/L. In embodiments, the usable quantity of MHCII/ligand complex expressed is between about 1 mg/L and about 70 mg/L. In embodiments, the usable quantity of MHCII/ligand complex expressed is between about 1 mg/L and about 60 mg/L. In embodiments, the usable quantity of MHCII/ligand complex expressed is between about 1 mg/L and about 50 mg/L. In embodiments, the usable quantity of MHCII/ligand complex expressed is between about 1 mg/L and about 40 mg/L. In embodiments, the usable quantity of MHCII/ligand complex expressed is between about 1 mg/L and about 30 mg/L. In embodiments, the usable quantity of MHCII/ligand complex expressed is between about 1 mg/L and about 20 mg/L. In embodiments, the usable quantity of MHCII/ligand complex expressed is between about 1 mg/L and about 10 mg/L. The amount may be any value or subrange within the recited ranges, including endpoints.

In embodiments, the signal range of MHCII/ligand complex detected by 2D LC-MS is between about 10³ to 10⁵ counts in the extracted ion chromatogram (EIC) of the peptide ligand. In some embodiments, the signal is about 10³ counts. In some embodiments, the signal is about 10⁴ counts. In some embodiments, the signal is about 10⁵ counts.

In embodiments, the peptide ligand has a peptide exchange yield of about 20% to about 100% in an exchange reaction with a peptide known to have high affinity for the allele. In embodiments, the peptide ligand has a peptide exchange yield of about 40% to about 100% in the exchange reaction. In embodiments, the peptide ligand has a peptide exchange yield of about 50% to about 100% in the exchange reaction. In embodiments, the peptide ligand has a peptide exchange yield of at least about 20% in the exchange reaction. In embodiments, the peptide ligand has a peptide exchange yield of at least about 30% in the exchange reaction. In embodiments, the peptide ligand has a peptide exchange yield of at least about 40% in the exchange reaction. In embodiments, the peptide ligand has a peptide exchange yield of at least about 50% in the exchange reaction. In embodiments, the peptide ligand has a peptide exchange yield of at least about 60% in the exchange reaction. In embodiments, the peptide ligand has a peptide exchange yield of at least about 70% in the exchange reaction. In embodiments, the peptide ligand has a peptide exchange yield of at least about 80% in the exchange reaction. In embodiments, the peptide ligand has a peptide exchange yield of at least about 90% in the exchange reaction. In embodiments, the peptide ligand has a peptide exchange yield of about 100% in the exchange reaction. The value may be any value or subrange within the recited ranges, including endpoints.

In embodiments, the MHCII complex has a higher affinity/IC₅₀ for the tagged peptide than the CLIP sequence. In some embodiments, the MHCII complex has a higher affinity for the tagged peptide than the CLIP sequence.

In embodiments, the MHCII/ligand complex contains an alpha chain, where the alpha chain is encoded by any one of the following loci: HLA-DP, HLA-DM, HLA-DOA, HLA-DOB, HLA-DQ and HLA-DR allele. In some embodiments, the alpha chain is encoded by the HLA-DP loci. In some embodiments, the alpha chain is encoded by the HLA-DM loci. In some embodiments, the alpha chain is encoded by the HLA-DOA loci. In some embodiments, the alpha chain is encoded by the HLA-DOB loci. In some embodiments, the alpha chain is encoded by the HLA-DQ loci. In some embodiments, the alpha chain is encoded by the HLA-DR loci.

In embodiments, the MHCII/ligand complex contains a beta chain, where the beta chain is encoded by any one of the following loci: HLA-DP, HLA-DM, HLA-DOA, HLA-DOB, HLA-DQ and HLA-DR allele. In some embodiments, the beta chain is encoded by the HLA-DP loci. In some embodiments, the beta chain is encoded by the HLA-DM loci. In some embodiments, the beta chain is encoded by the HLA-DOA loci. In some embodiments, the beta chain is encoded by the HLA-DOB loci. In some embodiments, the beta chain is encoded by the HLA-DQ loci. In some embodiments, the beta chain is encoded by the HLA-DR loci.

In embodiments, the MHCII/ligand complex is encoded by the HLA-DR allele and the ligand are selected from a list of HLA-DR and ligand sequences. In some embodiments, the HLA allele is HLA-DRB1*01:01 and the peptide ligand is PVSKARMATGALAQA (SEQ ID NO.: 1). In some embodiments, the HLA allele is HLA-DRB1*03:01 and the peptide ligand is PVSKMRMATGALAQA (SEQ ID NO.: 2). In some embodiments, the HLA allele is HLA-DRB1*04:01 and the peptide ligand is PVSKMRMATGALAQA (SEQ ID NO.: 2). In some embodiments, the HLA allele is HLA-DRB1*07:01 and the peptide ligand is PVSKMRMATGALAQA (SEQ ID NO.: 2). In some embodiments, the HLA allele is HLA-DRB1*08:01 and the peptide ligand is PVSKMRMATPLLMQA (SEQ ID NO.: 3). In some embodiments, the HLA allele is HLA-DRB1*11:01 and the peptide ligand is PVSKMRMATGALAQA (SEQ ID NO.: 2). In some embodiments, the HLA allele is HLA-DRB1*13:01 and the peptide ligand is PVSKMRMATPLLMQA (SEQ ID NO.: 3). In some embodiments, the HLA allele is HLA-DRB1*15:01 and the peptide ligand is PVSKARMATGALAQA (SEQ ID NO.: 1). In some embodiments, the HLA allele is HLA-DRB1*11:04 and the peptide ligand is PVSKMRMATGALAQA (SEQ ID NO.: 2).

In embodiments, an MHCII complex contains an MHC molecule containing an alpha chain and a beta chain, and a tagged peptide associated with the alpha chain and the beta chain.

In aspects is provided a composition including an MHCII complex (an MHC molecule containing an alpha chain and a beta chain and a peptide ligand associated with the alpha chain and the beta chain), and a test peptide. In embodiments, an MHCII complex contains: an MHC molecule containing an alpha chain and a beta chain, a peptide ligand, a test peptide, and a tagged peptide associated with the alpha chain and the beta chain. In embodiments, an MHCII complex contains: an MHC molecule containing an alpha chain and a beta chain, a peptide ligand, two or more test peptides, and a tagged peptide associated with the alpha chain and the beta chain. In some embodiments, the MHCII complex also contains a molecule that makes a detectable complex with the tagged peptide.

In embodiments, the major histocompatibility complex class II (MHCII)/ligand complex contains an MHCII molecule containing an alpha chain and a beta chain, and a ligand associated with the MHCII molecule. In some embodiments, the ligand is a peptide. In some embodiments, the ligand is a tagged peptide. In some embodiments, the ligand is a test peptide.

In embodiments, an MHCII complex contains an MHCII molecule containing an alpha chain and a beta chain, and a test peptide associated with the alpha chain and the beta chain. In some embodiments, the test peptide is a tumor antigen. In some embodiments, the test peptide is an autoantigen. In some embodiments, the test peptide is a neoantigen.

In embodiments, the test peptide is between 7 and 30 amino acid residues long. The length can be any value or subrange within the recited range, including endpoints. In some embodiments, the test peptide is 7 amino acid residues long. In some embodiments, the test peptide is 8 amino acid residues long. In some embodiments, the test peptide is 9 amino acid residues long. In some embodiments, the test peptide is 10 amino acid residues long. In some embodiments, the test peptide is 11 amino acid residues long. In some embodiments, the test peptide is 12 amino acid residues long. In some embodiments, the test peptide is 13 amino acid residues long. In some embodiments, the test peptide is 14 amino acid residues long. In some embodiments, the test peptide is 15 amino acid residues long. In some embodiments, the test peptide is 16 amino acid residues long. In some embodiments, the test peptide is 17 amino acid residues long. In some embodiments, the test peptide is 18 amino acid residues long. In some embodiments, the test peptide is 19 amino acid residues long. In some embodiments, the test peptide is 20 amino acid residues long. In some embodiments, the test peptide is 21 amino acid residues long. In some embodiments, the test peptide is 22 amino acid residues long. In some embodiments, the test peptide is 23 amino acid residues long. In some embodiments, the test peptide is 24 amino acid residues long. In some embodiments, the test peptide is 25 amino acid residues long. In some embodiments, the test peptide is 26 amino acid residues long. In some embodiments, the test peptide is 27 amino acid residues long. In some embodiments, the test peptide is 28 amino acid residues long. In some embodiments, the test peptide is 29 amino acid residues long. In some embodiments, the test peptide is 30 amino acid residues long.

In embodiments, the test peptide contains an amino acid sequence selected from: SEQ ID NO.: 1 to SEQ ID NO.: 16. In embodiments, the test peptide contains an amino acid sequence of SEQ ID NO.: 1.

### Method for Detecting MHCII/Ligand Complexes

In an aspect, provided herein, is a method of detecting binding of a major histocompatibility complex class II (MHCII) molecule to at test peptide, including: (a) providing a first composition including a peptide and a MHCII-ligand complex including (i) the MHCII molecule including an alpha chain and a beta chain and (ii) a ligand, wherein the ligand is bound to the alpha chain or the beta chain via a cleavable linker; (b) subjecting the first composition to a condition to cause cleavage of the cleavable linker; (c) incubating the first composition for a period of time sufficient to form a second composition, the second composition including the alpha chain, the beta chain, the ligand, free peptide, and/or a MHCII-peptide complex including the MHCII molecule and the peptide non-covalently bound to the MHCII molecule; and (d) determining whether the MHCII molecule is bound to the peptide.

In embodiments, whether the MHCII molecule binds to the test peptide is determined by measuring a level of MHCII/peptide complex in the second composition. In embodiments, the level of MHCII/peptide complex is measured by 2-dimensional liquid chromatography-mass spectrometry (2D LC/MS) of the second composition. In embodiments, the combination of HPLC and MS and/or 2D LC/MS is used to distinguish the MHCII molecule and the test peptide. In some embodiments, the free test peptide and/or ligand is removed from the second composition prior to 2D LC/MS. In some embodiments, the free test peptide is removed from the second composition prior to 2D LC/MS. In some embodiments, the free test peptide is removed from the second composition via size-exclusion chromatography (SEC). In some embodiments, the free test peptide is removed from the second composition via ion exchange.

In embodiments, the presence of the test peptide as determined by HPLC and MS indicates that the MHCII molecule is capable of binding to the test peptide. In embodiments, the presence of the test peptide as determined by HPLC and MS indicates that the test peptide has a higher affinity for the MHCII molecule compared to the ligand peptide.

The methods described herein can be run in multiplex. In embodiments, the first composition contains a plurality of the MHCII/ligand complex and at least two different test peptides. In embodiments, the first composition contains between 2 and about 1000 different test peptides. In embodiments, the first composition contains between 10 and about 500 different test peptides. In embodiments, the first composition contains between 50 and about 500 different test peptides. In embodiments, the first composition contains between 100 and about 500 different test peptides. In embodiments, the first composition contains between 10 and about 250 different test peptides. The number of test peptides may be any value or subrange within the recited ranges, including endpoints. In embodiments, the test peptides are distinguished from each other via mass spectrometry.

In embodiments, the test peptide(s) is present in the first composition at a ratio of between about 1000:1 and about 1:1000 test peptide to MHCII/ligand complex. In embodiments, the test peptide is present in the first composition at a ratio of between about 1000:1 and about 1:10 test peptide to MHCII/ligand complex. In embodiments, the test peptide is present in the first composition at a ratio of between about 1000:1 and about 1:1 test peptide to MHCII/ligand complex. In embodiments, the test peptide is present in the first composition at a ratio of between about 1000:1 and about 10:1 test peptide to MHCII/ligand complex. In embodiments, the test peptide is present in the first composition at a ratio of between about 1000:1 and about 100:1 test peptide to MHCII/ligand complex. The ratio may be any value or subrange within the recited ranges.

In embodiments, the MHCII molecule contains an HLA peptide encoded by an HLA-DP, HLA-DM, HLA-DO, HLA-DQ, or HLA-DR allele. In some embodiments, the HLA allele is HLA-DP. In some embodiments, the HLA allele is HLA-DM. In some embodiments, the HLA allele is HLA-DO. In some embodiments, the HLA allele is HLA-DQ. In some embodiments, the HLA allele is HLA-DR.

In embodiments, the MHCII molecule contains an HLA peptide encoded by an HLA-DR allele, and the ligand is a peptide. In embodiments, the HLA-DR allele is HLA-DRB. In embodiments, the HLA-DR allele is HLA-DRB1. In embodiments, the HLA-DRB allele is selected from HLA-DRB1*01:01, HLA-DRB1*03:01, HLA-DRB1*04:01, HLA-DRB1*07:01, HLA-DRB1*08:01, HLA-DRB1*11:01, HLA-DRB1*11:01, HLA-DRB1*13:01, or HLA-DRB1*15:01. In some embodiments, the allele is HLA-DRB1*01:01. In some embodiments, the allele is HLA-DRB1*03:01. In some embodiments, the allele is HLA-DRB1*04:01. In some embodiments, the allele is HLA-DRB1*07:01. In some embodiments, the allele is HLA-DRB1*08:01. In some embodiments, the allele is HLA-DRB1*11:01. In some embodiments, the allele is HLA-DRB1*13:01. In some embodiments, the allele is HLA-DRB1*15:01. In some embodiments, the HLA allele is HLA-DRB1*11:04.

In embodiments, the MHCII molecule/ligand complex contains a ligand that is a peptide. In embodiments, the ligand peptide is selected from the following: PVSKARMATGALAQA (SEQ ID NO.: 1), PVSKMRMATGALAQA (SEQ ID NO.: 2), or PVSKMRMATPLLMQA (SEQ ID NO.: 3). In some embodiments, the ligand peptide is PVSKARMATGALAQA (SEQ ID NO.: 1). In some embodiments, the ligand peptide is PVSKMRMATGALAQA (SEQ ID NO.: 2). In some embodiments, the ligand peptide is PVSKMRMATPLLMQA (SEQ ID NO.: 3).

In embodiments, the MHCII molecule/ligand complex contains a ligand that is a peptide, and the MHCII molecule is encoded by an HLA-DR allele. In some embodiments, the ligand peptide is PVSKARMATGALAQA (SEQ ID NO.: 1), and the HLA-DR allele is HLA-DRB1*01:01. In some embodiments, the ligand peptide is PVSKMRMATGALAQA (SEQ ID NO.: 2), and the HLA-DR allele is HLA-DRB1*01:01. In some embodiments, the ligand peptide is PVSKARMATGALAQA (SEQ ID NO.: 1), and the HLA-DR allele is HLA-DRB1*03:01. In some embodiments, the ligand peptide is PVSKMRMATGALAQA (SEQ ID NO.: 2), and the HLA-DR allele is HLA-DRB1*04:01. In some embodiments, the ligand peptide is PVSKMRMATGALAQA (SEQ ID NO.: 2), and the HLA-DR allele is HLA-DRB1*07:01. In some embodiments, the ligand peptide is PVSKMRMATPLLMQA (SEQ ID NO.: 3), and the HLA-DR allele is HLA-DRB1*08:01. In some embodiments, the ligand peptide is PVSKMRMATGALAQA (SEQ ID NO.: 2), and the HLA-DR allele is HLA-DRB1*11:01. In some embodiments, the ligand peptide is PVSKMRMATPLLMQA (SEQ ID NO.: 3), and the HLA-DR allele is HLA-DRB1*13:01. In some embodiments, the ligand peptide is PVSKARMATGALAQA (SEQ ID NO.: 1), and the HLA-DR allele is HLA-DRB1*15:01. In some embodiments, the HLA allele is HLA-DRB1*11:04 and the peptide ligand is PVSKMRMATGALAQA (SEQ ID NO.: 2).

In embodiments, the test peptide contains a sequence derived from an antigen, an autoantigen, or a neoantigen. In some embodiments, the test peptide contains a sequence derived from an antigen. In some embodiments, the test peptide contains a sequence derived from a neoantigen. In some embodiments, the test peptide contains a sequence derived from an autoantigen.

In embodiments, the test peptide is 7 to 30 amino acid residues long, as described above.

In embodiments, the test peptide contains an amino acid sequence selected from: SEQ ID NO.: 1 to SEQ ID NO.: 16.

In embodiments, provided herein, is a method for detecting the affinity of a test peptide for a major histocompatibility complex class II (MHCII) molecule, including: (a) providing a first composition including the test peptide; a tagged MHCII-binding peptide (tagged peptide); and a MHCII-ligand complex including (i) the MHCII molecule including an alpha chain and a beta chain and (ii) a ligand, wherein the ligand is bound to the alpha chain or the beta chain via a cleavable linker; (b) subjecting the first composition to a condition to cause cleavage of the cleavable linker; (c) incubating the first composition for a period of time sufficient form a second composition, the second composition including the alpha chain, the beta chain, the ligand, free test peptide, free tagged peptide, MHCII-tagged peptide complex including the tagged peptide associated with the MHCII molecule, and/or MHCII-test peptide complex including the test peptide associated with the MHCII molecule; and (d) determining whether the MHCII molecule is bound to the test peptide.

In embodiments, the first composition is incubated for at least 24 hours to 96 hours after initiation of the linker cleavage. In some embodiments, the first composition is incubated for 24 hours after initiation of linker cleavage. In some embodiments, the first composition is incubated for about 24 hours after initiation of linker cleavage. In some embodiments, the first composition is incubated for about 48 hours after initiation of linker cleavage. In some embodiments, the first composition is incubated for about 72 hours after initiation of linker cleavage. In some embodiments, the first composition is incubated for about 96 hours after initiation of linker cleavage. The incubation time may be any value or subrange within the recited ranges, including endpoints.

In embodiments, the second composition is contacted with an antibody that binds to said MHCII molecule. In embodiments, the antibody binds to the MHCII molecule. In embodiments, the antibody is immobilized on a solid surface.

In embodiments, the second composition is contacted with an antibody that binds to said MHCII molecule, and the second composition contains a tagged peptide. In embodiments, the tagged peptide is associated with the MHCII molecule.

In embodiments, the tagged peptide contains a label. In embodiments, the tagged peptide contains a fluorescent or bioluminescent label. In embodiments, the tagged peptide contains a biotin label.

In embodiments, the second composition is contacted with an antibody that binds to said MHCII molecule. In embodiments, the second composition contains an MHCII molecule and a tagged peptide. In embodiments, the tagged peptide contains a fluorescent or bioluminescent label. In embodiments, the tagged peptide contains a biotin label. In embodiments, the antibody is tagged with a fluorescent label, a bioluminescent label, or a substrate. In embodiments, the antibody is tagged with streptavidin-horseradish peroxidase conjugate.

In embodiments, the test peptide has a low affinity for the MHCII allele if the MHCII-tagged peptide complex is bound to the antibody.

In embodiments, the test peptide has a high affinity for the MHCII allele if the MHCII-tagged peptide complex is not bound to the antibody.

In embodiments, the affinity of the test peptide for the MHCII molecule is confirmed by a peptide exchange assay. In embodiments, the affinity of the test peptide for the MHCII molecule is determined compared to the affinity of a known peptide for the MHCII molecule.

In embodiments, the tagged peptide contains a human CLIP sequence.

In embodiments, the cleavable linker is a UV-cleavable linker or an enzyme-cleavable linker. In embodiments, the peptide linker sequence contains an enzyme cleavage site. In embodiments, the enzyme cleavage site is selected from: thrombin, enterokinase, Factor Xa, small ubiquitin-like modifier (SUMO) protease, tobacco etch-virus (TEV) protease, PreScission™ protease, rhinovirus 3C protease, Carboxypeptidase A, Carboxypeptidase B, dipeptidyl aminopeptidase (DAP), tobacco vein mottling virus protease (TVMV), and any variants thereof. In embodiments, the peptide linker sequence contains a non-natural amino acid. In embodiments, the non-natural amino acid is UV-cleavable. In some embodiments, the non-natural amino acid is selected from 2-nitrophenylglycine (NPG), expanded o-nitrobenzyl linker, o-nitrobenzylcaged phenol, o-nitrobenzyl caged thiol, 32 nitroveratryloxycarbonyl (NVOC) caged aniline, o-nitrobenzyl caged selenides, bis-azobenzene, coumarin, cinnamyl, spiropyran, 2-nitrophenylalanine (2-nF), and 3-amino-3-(2-nitrophenyl)propionic acid (ANP) amino acid analogs. In some embodiments, the non-natural amino acid is 3-amino-3-(2-nitrophenyl)propionic acid (ANP).

In embodiments, the peptide ligand satisfies one or more of the following criteria:
a) Produced at usable quantities during expression with an MHCII chain;
b) Is detected at high levels in 2D LCMS (after cleavage of linker); and/or
c) Has a peptide exchange yield of about 20% to 100% in an exchange reaction with a peptide known to have high affinity for the allele.

In embodiments, the test peptide is an antigen. In embodiments, the antigen is a cancer antigen. In embodiments, the antigen is a tumor-associated antigen. In embodiments, the antigen is a neoantigen. In embodiments, the antigen is an autoantigen.

In embodiments, the MHCII molecule comprises an HLA peptide encoded by an HLA-DP allele, an HLA-DM allele, an HLA-DOA allele, an HLA-DOB allele, an HLA-DQ allele, or an HLA-DR allele. In some embodiments, the HLA allele is HLA-DP. In some embodiments, the HLA allele is HLA-DM. In some embodiments, the HLA allele is HLA-DO. In some embodiments, the HLA allele is HLA-DQ. In some embodiments, the HLA allele is HLA-DR.

In embodiments, the MHCII molecule contains an HLA peptide encoded by an HLA-DR allele, and the ligand is a peptide. In embodiments, the HLA-DR allele is HLA-DRB. In embodiments, the HLA-DR allele is HLA-DRB1. In embodiments, the HLA-DRB allele is selected from HLA-DRB1*01:01, HLA-DRB1*03:01, HLA-DRB1*04:01, HLA-DRB1*07:01, HLA-DRB1*08:01, HLA-DRB1*11:01, HLA-DRB1*11:04, HLA-DRB1*13:01 or HLA-DRB1*15:01. In some embodiments, the allele is HLA-DRB1*01:01. In some embodiments, the allele is HLA-DRB1*03:01. In some embodiments, the allele is HLA-DRB1*04:01. In some embodiments, the allele is HLA-DRB1*07:01. In some embodiments, the allele is HLA-DRB1*08:01. In some embodiments, the allele is HLA-DRB1*11:01. In some embodiments, the allele is HLA-DRB1*13:01. In some embodiments, the allele is HLA-DRB1*15:01.

In embodiments, the MHCII molecule/ligand complex contains a ligand that is a peptide. In embodiments, the ligand peptide is selected from the following: PVSKARMATGALAQA (SEQ ID NO.: 1), PVSKMRMATGALAQA (SEQ ID NO.: 2), or PVSKMRMATPLLMQA (SEQ ID NO.: 3). In some embodiments, the ligand peptide is PVSKARMATGALAQA (SEQ ID NO.: 1). In some embodiments, the ligand peptide is PVSKMRMATGALAQA (SEQ ID NO.: 2). In some embodiments, the ligand peptide is PVSKMRMATPLLMQA (SEQ ID NO.: 3).

In embodiments, the MHCII molecule/ligand complex contains a ligand that is a peptide, and the MHCII molecule is encoded by an HLA-DR allele. In some embodiments, the ligand peptide is PVSKARMATGALAQA (SEQ ID NO.: 1), and the HLA-DR allele is HLA-DRB1*01:01. In some embodiments, the ligand peptide is PVSKMRMATGALAQA (SEQ ID NO.: 2), and the HLA-DR allele is HLA-DRB1*01:01. In some embodiments, the ligand peptide is PVSKARMATGALAQA (SEQ ID NO.: 1), and the HLA-DR allele is HLA-DRB1*03:01. In some embodiments, the ligand peptide is PVSKMRMATGALAQA (SEQ ID NO.: 2), and the HLA-DR allele is HLA-DRB1*04:01. In some embodiments, the ligand peptide is PVSKMRMATGALAQA (SEQ ID NO.: 2), and the HLA-DR allele is HLA-DRB1*07:01. In some embodiments, the ligand peptide is PVSKMRMATPLLMQA (SEQ ID NO.: 3), and the HLA-DR allele is HLA-DRB1*08:01. In some embodiments, the ligand peptide is PVSKMRMATGALAQA (SEQ ID NO.: 2), and the HLA-DR allele is HLA-DRB1*11:01. In some embodiments, the ligand peptide is PVSKMRMATPLLMQA (SEQ ID NO.: 3), and the HLA-DR allele is HLA-DRB1*13:01. In some embodiments, the ligand peptide is PVSKARMATGALAQA (SEQ ID NO.: 1), and the HLA-DR allele is HLA-DRB1*15:01. In some embodiments, the HLA allele is HLA-DRB1*11:04 and the peptide ligand is PVSKMRMATGALAQA (SEQ ID NO.: 2).

### Multiplex Epitope Mapping

Multiplex epitope mapping can be used to determine the most likely peptide to bind an MHCII allele (e.g., most likely to bind in vivo) from a given antigen. For example, a neoantigen containing a mutation (point mutation, indel, etc.) could be presented by MHCII as any of a number of peptides. Each possible peptide will have a different affinity for a given MHCII allele. The methods described herein allow competition binding and analysis in a multiplex manner to map long mutant peptides in a single run. The different peptides compete for binding of the MHCII molecule, allowing the “best binder” (peptide having the highest affinity to the MHCII allele under the conditions used) to be determined. The relative binding affinities of the various peptides may also be determined. The best binder has a high likelihood of being presented by MHCII on the surface of a cell. As would be understood by one of skill in the art, this method may use any of the compositions, MHCII molecules, MHCII-ligand complexes, and methods described elsewhere in this disclosure.

In an aspect, provided herein, is a method for multiplex epitope mapping for a major histocompatibility complex class II (MHCII) allele. In embodiments, the method includes:
a) providing a first composition including a plurality of test peptides, and a plurality of MHCII-ligand complexes, each MHCII-ligand complex having (i) a MHCII molecule including an alpha chain and a beta chain and (ii) a ligand, wherein the ligand is bound to the alpha chain or the beta chain via a cleavable linker;
b) subjecting the first composition to a condition to cause cleavage of the cleavable linker;
c) incubating the first composition for a period of time sufficient to form a second composition, the second composition including the alpha chain, the beta chain, the ligand, free test peptide, and/or a plurality of MHCII-test peptide complexes, each MHCII-test peptide complex including the MHCII molecule and a test peptide non-covalently bound thereto; and
d) determining whether one or more of the plurality of test peptides is bound to the MHCII molecule in the second composition.

In embodiments, each test peptide in the plurality of test peptides is present in a sub-stoichiometric amount compared to the amount of MHCII molecule in the first composition. Without being bound by theory, it is expected that this will allow kinetic competition between the test peptides, but not thermodynamic competition. This is expected to allow determination of which test peptides are capable of binding, but not necessarily the test peptide that is the best binder.

In embodiments each test peptide in the plurality of test peptides is present in excess compared to the amount of MHCII molecule in the first composition. Without being bound by theory, it is expected that this will allow determination of the test peptide that binds best (e.g., with highest affinity) to the MHCII molecule.

In embodiments, the plurality of test peptides comprises overlapping peptides of an antigen. In embodiments, the plurality of test peptides contain sequential peptides from a single protein. In embodiments, the antigen is a neoantigen, a tumor-associated antigen, or an autoantigen. In embodiments, the antigen is a neoantigen. In embodiments, the antigen is a tumor-associated antigen. In embodiments, the antigen is an autoantigen. The phrase “overlapping peptides of an antigen” as used herein refers to peptides that correspond to a portion of an antigen or potential antigen, each peptide (or subset of peptides) corresponding to a slightly different portion of the antigen. For example, where an antigen comprises the sequence ABCDEFGH, overlapping peptides of the antigen may include ABC, BCD, CDE, DEF, EFG, FGH, and/or ABCD, BCDE, CDEF, DEFG, EFGH, and so on.

In embodiments, the length of each test peptide is between 7 amino acids and 30 amino acids. The length can be any value or subrange within the recited range, including endpoints. In some embodiments, the test peptide is 7 amino acid residues long. In some embodiments, the test peptide is 8 amino acid residues long. In some embodiments, the test peptide is 9 amino acid residues long. In some embodiments, the test peptide is 10 amino acid residues long. In some embodiments, the test peptide is 11 amino acid residues long. In some embodiments, the test peptide is 12 amino acid residues long. In some embodiments, the test peptide is 13 amino acid residues long. In some embodiments, the test peptide is 14 amino acid residues long. In some embodiments, the test peptide is 15 amino acid residues long. In some embodiments, the test peptide is 16 amino acid residues long. In some embodiments, the test peptide is 17 amino acid residues long. In some embodiments, the test peptide is 18 amino acid residues long. In some embodiments, the test peptide is 19 amino acid residues long. In some embodiments, the test peptide is 20 amino acid residues long. In some embodiments, the test peptide is 21 amino acid residues long. In some embodiments, the test peptide is 22 amino acid residues long. In some embodiments, the test peptide is 23 amino acid residues long. In some embodiments, the test peptide is 24 amino acid residues long. In some embodiments, the test peptide is 25 amino acid residues long. In some embodiments, the test peptide is 26 amino acid residues long. In some embodiments, the test peptide is 27 amino acid residues long. In some embodiments, the test peptide is 28 amino acid residues long. In some embodiments, the test peptide is 29 amino acid residues long. In some embodiments, the test peptide is 30 amino acid residues long.

In embodiments, MHCII molecule binding to the one or more of the plurality of test peptides is determined by measuring a level of each test peptide bound to the MHCII molecule in the second composition. In embodiments, the level of each test peptide bound to the MHCII molecule is measured by 2-dimensional liquid chromatography-mass spectrometry (2D LC/MS) of the second composition.

In embodiments, the method further includes performing high-performance liquid chromatography (HPLC) and mass spectrometry (MS) to distinguish the MHCII molecule and the test peptides. In embodiments, the test peptides are distinguished from each other by mass spectrometry based on the mass of each test peptide.

In embodiments, 2D LC/MS includes removing the free test peptide and/or ligand from the second composition. In embodiments, the free test peptide is removed from the second composition by size exclusion chromatography or ion exchange chromatography.

In embodiments, presence of a given test peptide as determined by HPLC and MS indicates that the given test peptide is capable of binding to the MHCII molecule. In embodiments, the relative levels of the test peptides bound to the MHCII molecules indicate the relative affinities of the test peptides for the MHCII molecule. In embodiments, a lower level of a first test peptide bound to the MHCII molecules compared to a second test peptide indicates that the first test peptide has a lower likelihood of binding the MHCII molecule on the surface of a cell than the second test peptide. In embodiments, a higher level of a second test peptide bound to the MHCII molecules compared to a first test peptide indicates that the second test peptide has a higher likelihood of binding the MHCII molecule on the surface of a cell than the first test peptide.

In embodiments, one or more of the test peptides determined to have a higher affinity than at least one other test peptide is further analyzed. For example, the one or more test peptides may be analyzed

In embodiments, the peptide that binds at least one major histocompatibility complex class II (MHCII) molecule, the peptide containing an amino acid sequence selected from: SEQ ID NO.: 1 to SEQ ID NO.: 16.

### EXAMPLES

One skilled in the art would understand that descriptions of making and using the particles described herein is for the sole purpose of illustration, and that the present disclosure is not limited by this illustration.

### Example 1. CLIP Variant Effects on Peptide Exchange

Variants of CLIP peptides that are genetically encoded and fused to the N-terminus of the HLA-DR beta chain have been identified that allow robust expression and peptide exchange to be possible across a panel of HLA-DR alleles. The CLIP peptides can be released by cleavage using either a thrombin or TEV cleavage site and under appropriate conditions, the CLIP peptide is released and can be exchanged by a candidate antigen peptide. The extent and validation of peptide exchange can be determined by either a competition ELISA assay or 2D-LCMS assay described in the second component of this invention.

The HLA-CLIP peptide fusion proteins were expressed in HEK-293 or CHO cells. The expressed HLA fusion protein containing a protein-CLIP peptide linker with a thrombin recognition site was enzymatically processed using a thrombin-agarose digest. Briefly, ˜3 mg/mL of the uncleaved pMHCII fusion protein complex was buffer exchanged into 25 mM Tris, pH 8.0, 2 mM NaN₃ using a pre-equilibrated Zeba™ spin column. The exchanged complex was incubated in the presence of thrombin-agarose at room temperature with mixing until the majority of the pMHCII fusion protein had been cleaved, as determined by LC/MS.

The expressed HLA fusion protein containing a protein-CLIP peptide linker with a TEV (tobacco etch virus) protease recognition site was enzymatically processed using a TEV digest. Briefly, ˜3 mg/mL of the uncleaved pMHCII fusion protein complex at pH 6-8 was incubated with ˜30 μg TEV protease at room temperature. The pMHCII fusion protein was digested until the majority had been cleaved, as determined by LC/MS.

The pMHCII fusion protein complexes were then purified by size-exclusion chromatography (SEC). Briefly, sodium chloride (NaCl) was added to the pMHCII fusion protein complex solution to a final concentration of 300 mM. The solution was concentrated to between 0.5-1.0 mg/mL, and injected onto an FPLC equipped with an S-200 SEC column (GE Healthcare) equilibrated with phosphate buffered saline (PBS), 150 mM NaCl, 2 mM NaN₃. The column was run at 0.5 mL/min, and the protein was collected after determining concentration in the eluent by UV-Vis spectroscopy (Implen NanoPhotometer). The eluted, purified protein was further concentrated by spin filter (Amicon, Millipore) to a concentration less than ˜5 mg/mL.

The pMHCII fusion protein complexes were exchanged with a test peptide(s). Briefly, a master mix of ˜18 μM CLIP-MHCII in 40 mM sodium acetate, pH 5.0, 150 mM NaCl, 4 mM EDTA, 2 mM NaN₃ was prepared (with addition of 0.5 mM TCEP for cysteine containing peptides). For a 50 μL reaction, 5 μL of 4 mM peptide(s) was added to 45 μL of master mix, mixed, and incubated for 60 min at 37° C. The exchange reaction was quenched by addition of an equal volume of 50 mM Tris pH 8, 150 mM NaCl (final pMHCII concentration=8 μM). The peptide exchange mixture was centrifuged at 14,000×g for 2 minutes to remove insoluble peptide or protein aggregates. The mixture was stored at 4° C. and protected from light before further analysis.

MHCII: Although use of a lower affinity peptide for HLA-DRB1*01:01 has been reported, a novel CLIP peptide variant that can be used that is superior for some HLA-DR alleles that could not be identified without experimental validation. Furthermore, these variants allow robust protein expression while also enabling peptide exchange.

### Example 2. Biotinylated Tetramer Formation

The pMHCII fusion protein complexes were biotinylated via BirA biotin protein ligase. Briefly, a reaction mixture containing 100 mM magnesium acetate, 10 mM ATP, 5 mM biotin, and ˜3 mg/mL pMHCII was prepared. The reaction was initiated by adding 1.3 mg/mL FLAG-BirA, and incubated at room temperature overnight.

The biotinylated pMHCII fusion protein complexes were then purified by size-exclusion chromatography (SEC). Briefly, sodium chloride (NaCl) was added to the biotinylated pMHCII fusion protein complex solution to a final concentration of 300 mM. The solution was concentrated to between 0.5-1.0 mg/mL, and injected onto an FPLC equipped with an S-200 SEC column (GE Healthcare) equilibrated with phosphate buffered saline (PBS), 150 mM NaCl, 2 mM NaN₃. The column was run at 0.5 mL/min, and the protein was collected after determining concentration in the eluent by UV-Vis spectroscopy (Implen NanoPhotometer). The eluted, purified protein was further concentrated by spin filter (Amicon, Millipore) to a concentration less than ˜5 mg/mL.

The biotinylated, pMHCII fusion protein complexes were exchanged with a test peptide(s). Briefly, a master mix of ˜18 μM CLIP-MHCII in 40 mM sodium acetate, pH 5.0, 150 mM NaCl, 4 mM EDTA, 2 mM NaN₃ was prepared (with addition of 0.5 mM TCEP for cysteine containing peptides). For a 50 μL reaction, 5 μL of 4 mM peptide(s) was added to 45 μL of master mix, mixed, and incubated for 60 min at 37° C. The exchange reaction was quenched by addition of an equal volume of 50 mM Tris pH 8, 150 mM NaCl (final pMHCII concentration=8 μM). The peptide exchange mixture was centrifuged at 14,000×g for 2 minutes to remove insoluble peptide or protein aggregates. The purified peptide-exchanged, biotinylated pMHCII were added to streptavidin-fluorophore (2:1 pMHCII to streptavidin-phyrocoerythrin) and mixed thoroughly. The mixture was stored at 4° C. and protected from light before analysis by analytical SEC.

The purified, biotinylated, peptide-exchanged pMHCII fusion protein-streptavidin complexes were evaluated to determine the extent of the biotinylation in each mixture. Briefly, the following mixtures were analyzed per injection via analytical SEC: 2×pMHCII: 20 μL of 8 μM pMHCII-biotin, 10 μL PBS, 2×pMHCII, 1×PE-SAv: 20 μL of 8 μM pMHCII-biotin, 5.5 μL of 0.2 mg/mL streptavidin-phyrocoerythrin (SAv-PE, BioLegend), 4.5 μL PBS, and 1×PE-SAv: 10 μL of 8 μM pMHCII-biotin, 5.5 μL of 0.2 mg/mL SAv-PE, 14.5 μL PBS. The mixtures were measured on an HPLC equipped with an analytical SEC column (TSKgel G3000SWXL) equilibrated with 1×PBS, running at a flow rate of 0.65 mL/min.

### Example 3. 2D LC-MS Assay to Identify MHCII Peptide Binders

This assay identifies peptide binders after a peptide exchange assay for MHCII complexes. Mass spectrometry based analysis of the peptide exchange process depends on first separating out the MHCII complex from free peptide in solution prior to analysis. Here is described a 2D LC-MS analysis method where the sample is first run on an SEC column and then only the peak that corresponds to the MHCII complex is injected onto the second HPLC column for Mass Spec analysis. This allows for complete analysis of the MHCII reagent in a single step. This process can be used to identify a single peptide binder at a time or binders within a larger pool of peptides.

The first dimension for the 2D LC-MS analysis is size exclusion chromatography. The configuration used here were an HPLC (Agilent 1200 Series) equipped with a Zenix SEC-100, 3 μm, 100 Å, 4.6×50 mm column, run at a flow rate of 0.4 mL/min at 30° C. The column was equilibrated and run in 25 mM Tris pH 8, 150 mM NaCl, 2 mM NaN₃. The collected eluent eluted between 2.43-2.88 min using the heart-string cut-off, and was subsequently diverted to the second dimension in the analysis.

The second dimension 2D LC-MS analysis is reversed-phase chromatography. The configuration used here was an HPLC equipped with a PLRP-S, 8 μm, 1000 Å, 2.1×50 mm reversed phase column (Agilent) equilibrated with 5% buffer B running at a flow rate of 0.55 mL/min at a column temperature of 80° C. Additionally, the cycle modulation time was 8 min, loop volume was 180 μL, and diverter valve switch time was 2 min. A gradient was used to elute the injected peptides and protein, where buffer A was 0.05% trifluoroacetic acid in water, and buffer B was 0.05% trifluoroacetic acid in acetonitrile. The gradient was as follows: 0.00 min-5.0% buffer B, 2.00 min-5.0% buffer B, 6.70 min-50.0% buffer B, 6.71 min-95.0% buffer B, 7.60 min-95% buffer B, and 7.61 min-5.0% buffer B.

The eluent was injected onto an Agilent Technologies 6224 TOF LC/MS with extended dynamic range and a dual ESI ion source. Briefly, the mass spectrometer settings were: positive ion polarity, carrier gas temperature, 350° C., drying gas flow rate of 13 L/min, nebulizer pressure of 45 psi, skimmer voltage of 65 V, OCT 1RF VPP at 750 V, and Vcap at 4000 V The acquisition mass range was a min 100 m/z and max 3000 m/z the acquisition rate was 1.03 spectra/s, 970.9 ms/spectrum, and the transients/spectrum, 9679.

Sample type and resulting absorbance/count ranges were as follows. Briefly, 20 μL of 8 μM (˜0.5 mg/mL) peptide-MHCII was injected per LC-MS run. For 20 μL of unexchanged 8 μM CLIP-MHCII injections, the absorbance at 280 nm in the first dimension was in the range of 150-200 mAU, and in the second dimension, the EIC of CLIP (M+H, M+2H, M+3H) was ˜10\hat{ }5 counts. For 20 μL of >95% exchanged peptide-MHCII injected, the EIC of CLIP was ˜10\hat{ }3 counts. Partial exchange caused the EIC to drop to 10\hat{ }4-10\hat{ }3 counts depending on the amount of protein recovered from the peptide exchange reaction after centrifugation. The low CLIP-MHCII samples were injected at a concentration of 16 μM MHCII and 400 μM peptide.

### Example 4. Identification of Mutation-Specific CD4 T Cells

Mutations identified from the MC38 colorectal carcinoma mouse cell line (Yadav M. et al, Nature 2014) have been screened for their capability to induce mutation-specific T cell responses upon vaccination in animal studies using a 24 amino-acid (AA) length mutated peptide (Capietto A H et al., JEM, 2020 and unpublished data). Peptide libraries from immunogenic mutations were then designed and synthesize (Genscript) with 4 overlapping 15 amino-acid peptides including the mutation. Each peptide was tested for binding to IAb molecule using the above described 2D-LCMS method. In order to confirm specific T cell recognition of the binding peptides, healthy WT C57Bl/6 mice (Jackson Laboratory) were vaccinated with the 24 AA length peptide (n=3, 100 μg/mouse; intraperitoneal injection) and adjuvants (poly(IC), 100 μg/mouse, Invitrogen and anti-CD40 antibody, 50 μg/mouse, Genentech) on days 0 and 10. Spleens were harvested 5 days after the last vaccination and processed to obtain a single cell suspension. T cell responses were then determined using mouse IFN-gamma ELISpot assays (Biotechne) with total splenocytes (5 10\hat{ }5 cells) incubated overnight with each 15 AA peptide (1 μg/mL), the 24 AA peptide (10 μg/mL) or no peptide as control in complete RPMI 1640 media (10% FBS, 1% penicillin/streptomycin, 1% glutamate). IFNg spots were revealed following the manufacturer instructions. Finally, peptides showing both binding to IAb molecule (2D-LCMS) and immunogenicity (ELISpot) were used to form tetramers and tested for staining by flow cytometry on splenocytes (20 μg/mL in 100 μL of FACS buffer, 1 hour at room temperature (RT) in the dark) from vaccinated and non-vaccinated mice as control.

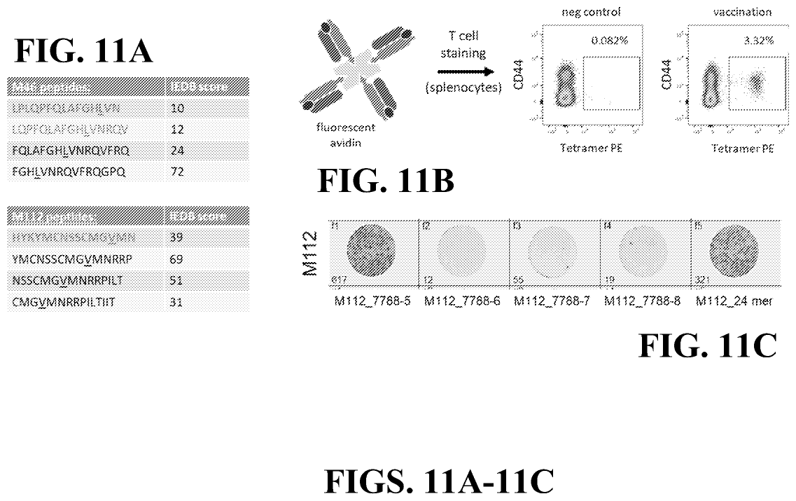

The results from vaccination of mice from mutation 112 from MC38 tumor cells a shown as an example in FIGS. 11A-11C. FIG. 11A shows the overlapping peptide library. FIG. 11B shows a tetramer schematic with peptide 7788-5 as the ligand with PE fluorophore labeling, and the resulting flow cytometry (FACS) analysis. Splenocytes from whole spleen of vaccinated mice were stained with pIAb (7788-5) tetramer for 1 hour at RT in the dark in FACS buffer (20 μg/mL, 100 μL), washed twice with 200 μL of FACS buffer and stained with anti-PE microbeads (Miltenyi) following the manufacturer instructions. Tetramer-positive cells were then enriched through a magnetic LS column (Miltenyi) and stained for surface antibodies and live/dead staining at 4° C. for 20 minutes. Cells were then washed twice with 200 μL and acquired on a BD-Symphony Flow cytometer to detect activated (CD44+) tetramer+ CD4 T cells. Total splenocytes from non-vaccinated mice were used as negative control. FIG. 11C shows the resulting ELISpot assay for an in vivo vaccination in mice of peptides M112_7788-5, M112_7788-6, M112_7788-7, M112-7788-8, and M112_24 mer. Only peptide 7788-5 and the 24 AA peptide showed IFN-gamma spots after recognition by specific CD4 T cells from vaccinated mice in ELISpot assay.

### Example 5. Multiplexed Epitope Mapping

This mass spectrometry-based assay identifies peptide binders after a peptide exchange assay for MHCII complexes. Described here is an automated immunoprecipitation approach to enrich for exchanged peptides forming complexes with biotinylated recombinant MHCII proteins. Peptide-MHCII complexes are loaded onto Agilent AssayMAP Streptavidin cartridges (cat G5496-60010, Agilent, Santa Clara, CA). Using the Agilent Bravo AssayMAP system, peptides are acid eluted from streptavidin bound peptide-MHCII complex. Peptide eluants are loaded onto a reversed-phase C18 column followed by gradient elution directly into a Thermo tribrid orbitrap mass spectrometer for downstream LC-MS/MS analysis. Peaks from mass spectrometric analysis corresponding to m/z of interest are selected to confirm their presence and area under the curve values are calculated to determine binding affinity relative to that of the control sample signal where all peptides were present at 1-1 molar ratio.

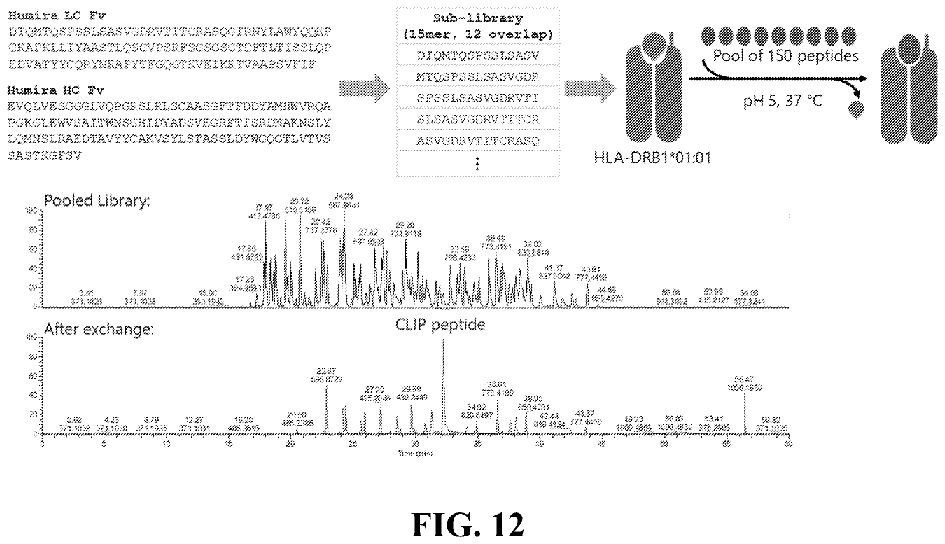

FIG. 12 shows multiplexed analysis of peptide binding of a 150 peptide library from Humira and Remicade variable domains in a single exchange.

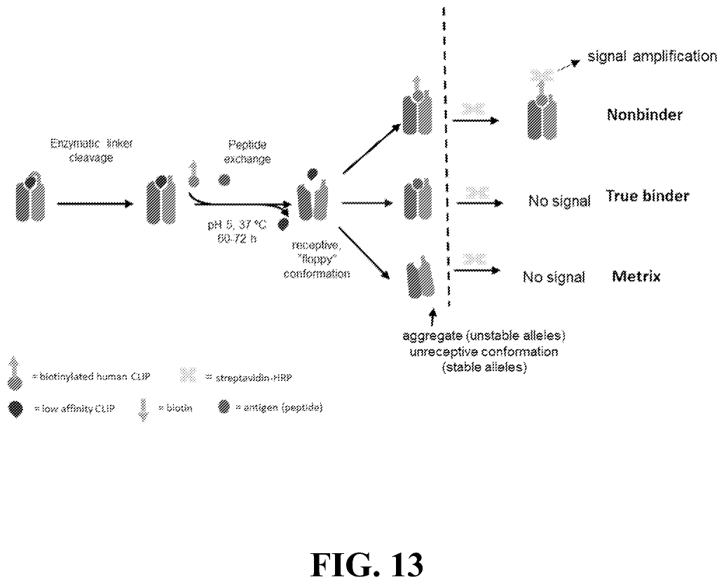

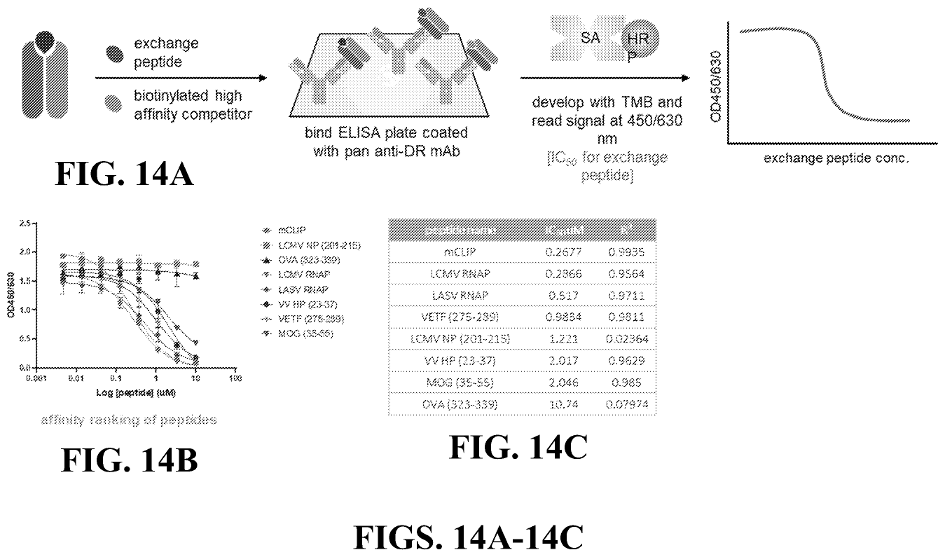

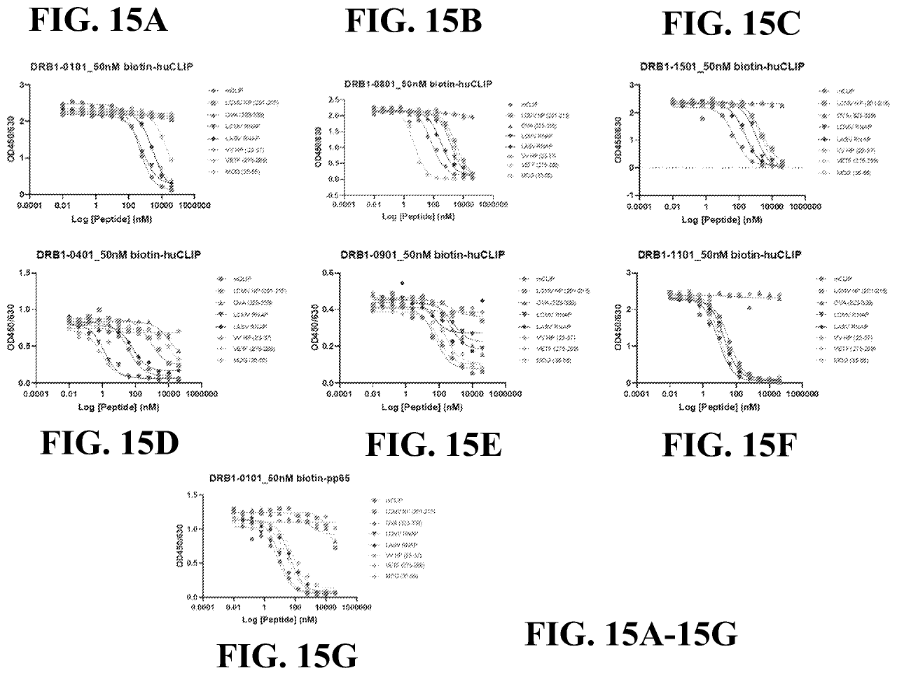

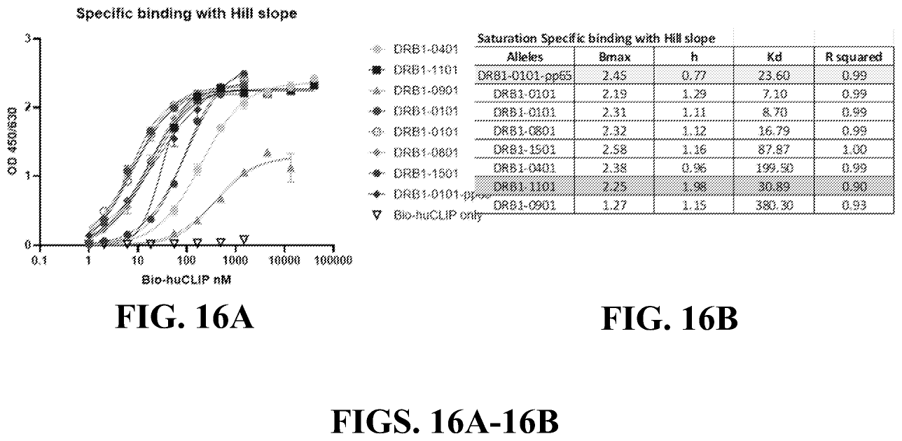

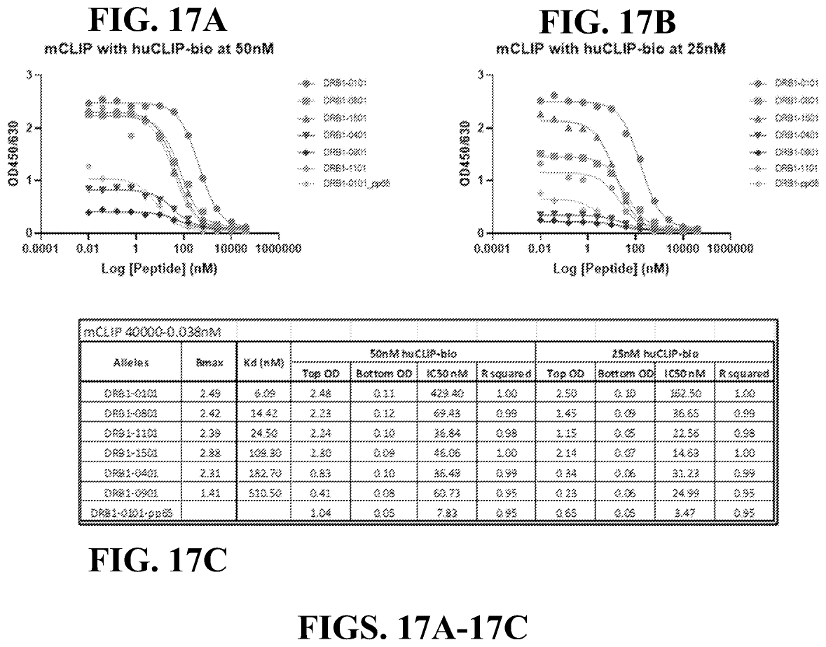

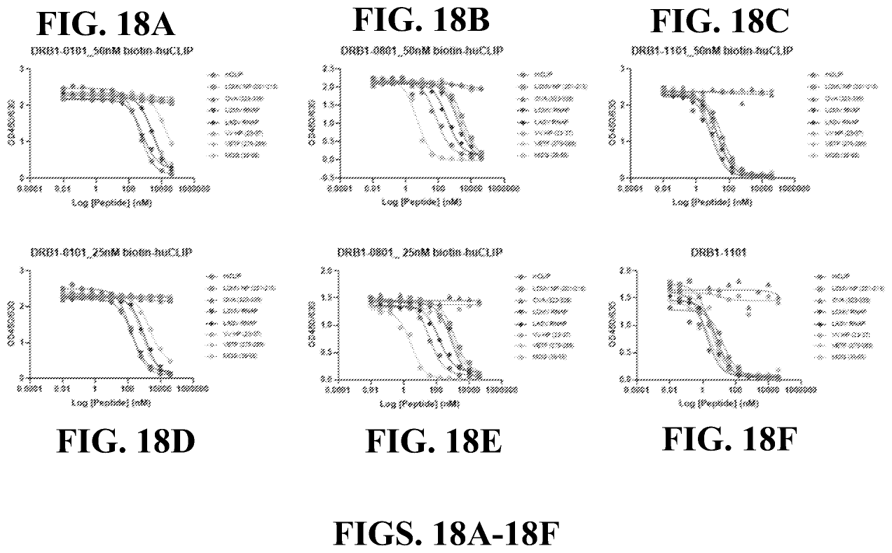

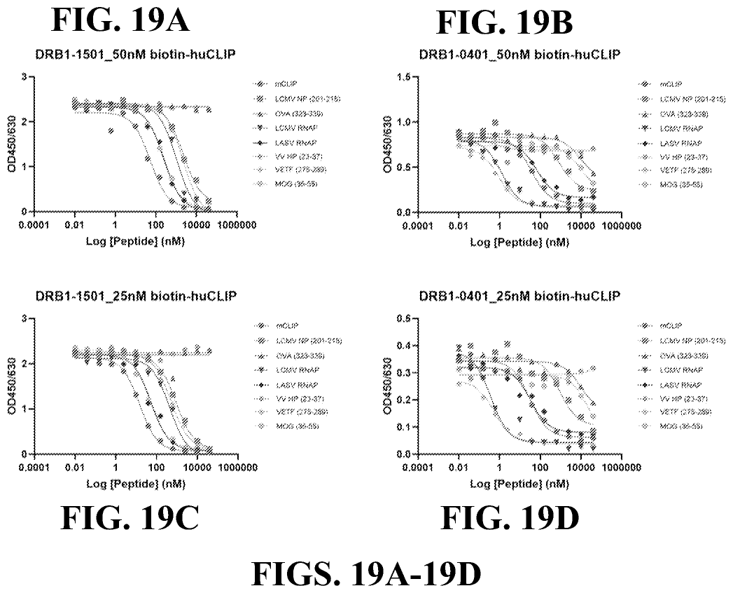

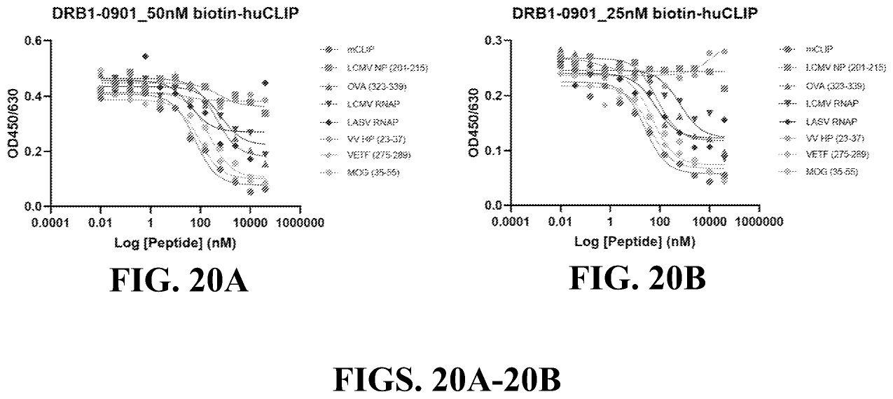

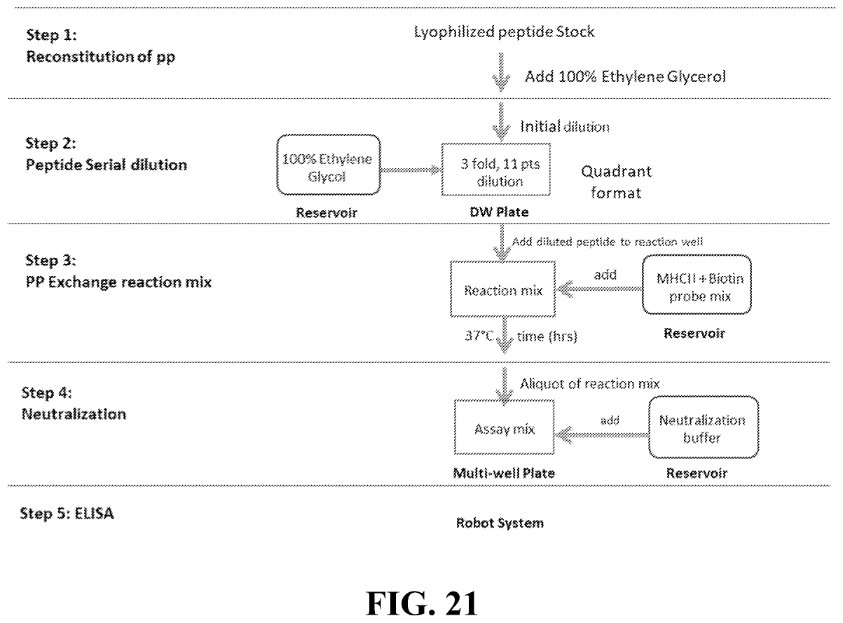

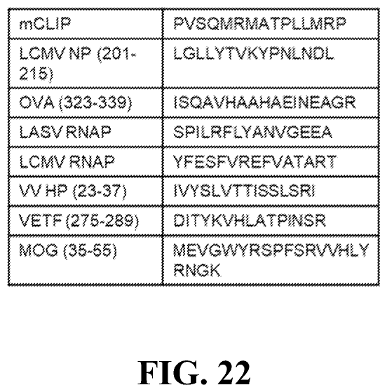

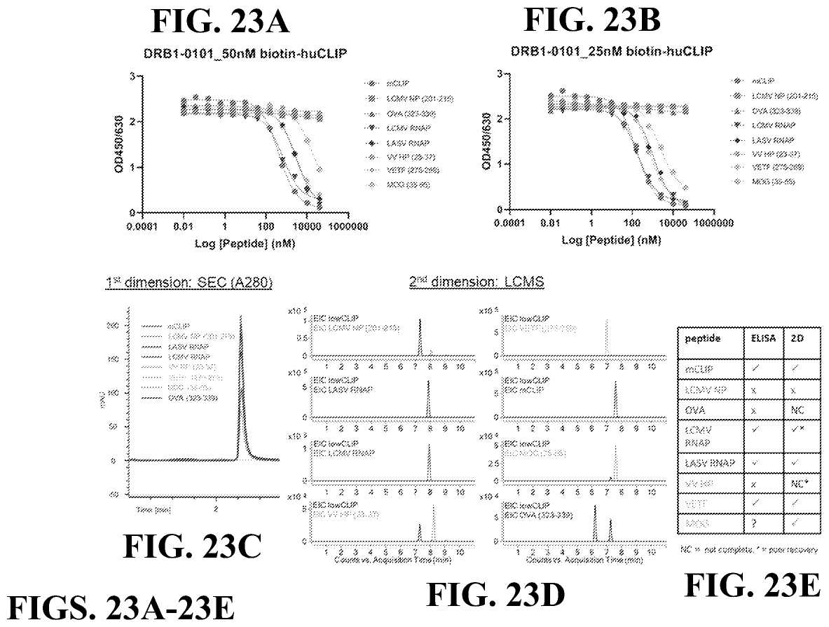

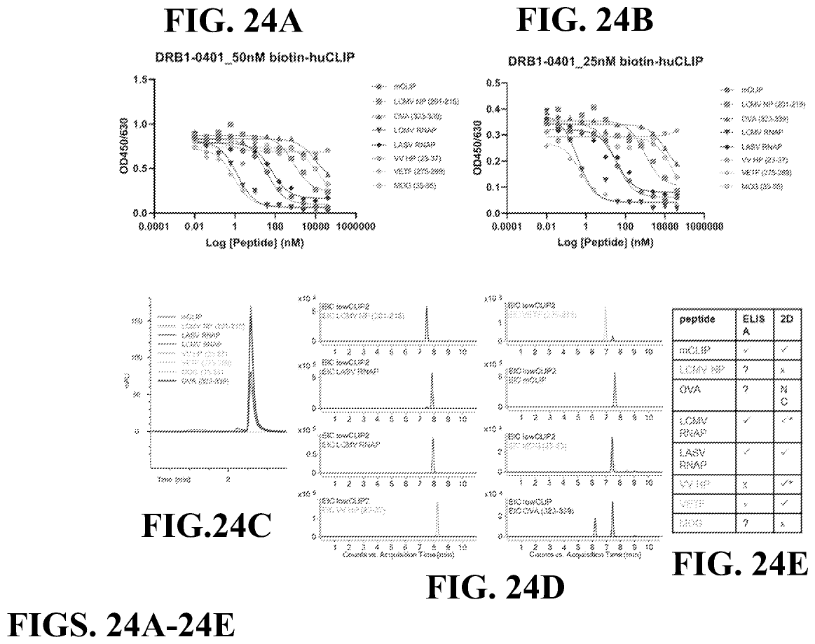

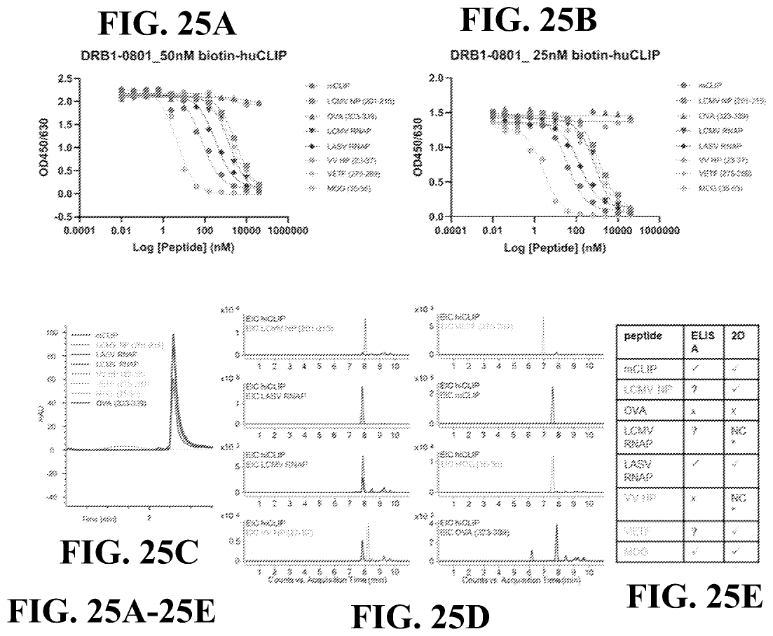

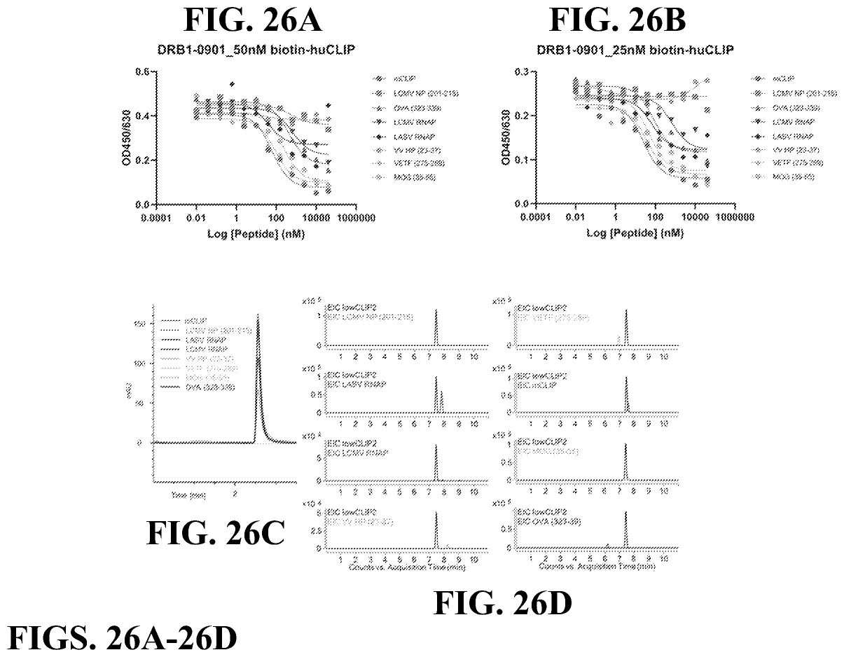

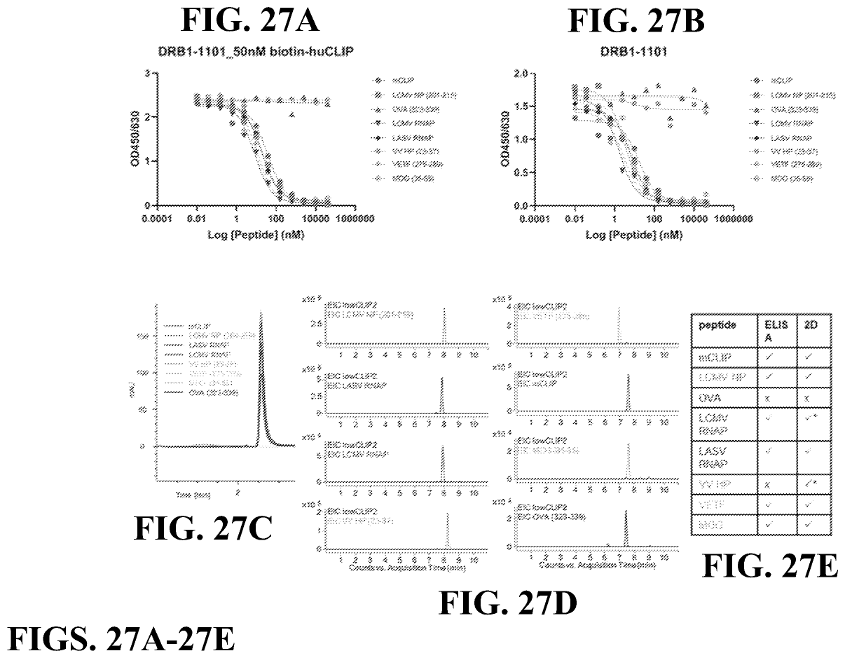

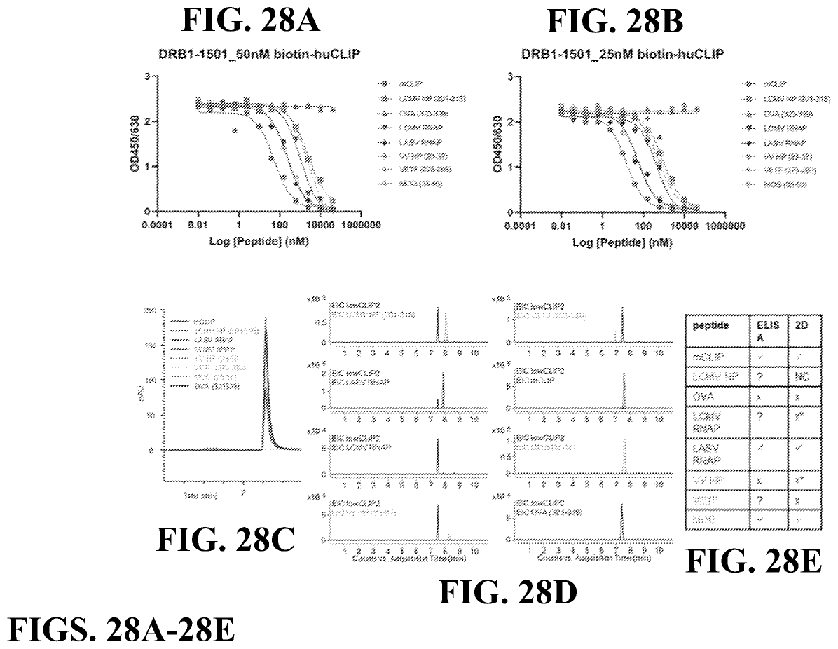

FIG. 13 is a schematic of a competitive peptide exchange assay. A low affinity containing peptide is covalently attached to an MHCII molecule, via a cleavable linker. The linker is cleaved in the presence of a pool of labeled, high affinity peptide and a test peptide(s), and the low affinity peptide is exchanged. The three outcomes illustrated are: MHCII molecule binds the high-affinity peptide, and is detected, the MHCII molecule binds the test peptide(s), and is not detected, and/or the MHCII complex becomes unable to bind a peptide, and is not detected.

### Example 6. High Throughput ELISA Competition Assay

Peptide Exchange: A master mix containing 110 nM of MHCII/low affinity CLIP and 56 nM or 28 nM of biotinylated high affinity huCLIP (huCLIP-bio) was prepared in exchange buffer (40 mM sodium acetate, 150 mM NaCl, 4 mM EDTA, pH 5.0). In a 384 well plate, a peptide stock at 4 mM in 100% ethylene glycerol (EG) was initially diluted to 400 μM followed by three fold, 11 point serial dilution in 100% ethylene glycerol on a BioMek system (Beckman Dickson, i7) to generate the 10× intermediate working solutions with a concentration range of 400000 nM to 0.38 nM. Ethylene glycerol (without peptide) was used as the negative control. The peptide exchange was carried out in a 384 well deep well plate by transferring 90 μL of master mix and 10 μL of pre-titrated peptide in each well. The final peptide reaction mix contained 100 nM MHCII/low affinity CLIP, 50 or 25 nM huCLIP-bio and test peptide ranged from 40000 nM to 0.038 nM. The plate then was sealed and incubated at 37° C. for 48 to 70 hours to allow the peptide exchange. After completing the incubation, plate was spin down and the reaction mix was neutralized by transferring 50 μL/well of the reaction mix to the well of a new 384 well plate containing 50 μL/well of neutralization buffer (400 mM tris, 150 mM NaCl, pH 8.0).

Competitive ELISA: A 384 well Maxisorp plate (Thermo Fisher, Nunc #464718) was coated with 25 μL/well of a mouse IgG2a anti-HLA-DR1 L243 monoclonal antibody (Genentech, PUR85601) at 10 μg/mL in coating buffer (0.05M sodium carbonate, pH 9.6). After incubating overnight at 4° C. and washing the plate three times with wash buffer (PBS buffer with 0.05% Tween 20), 50 L/well of block buffer (PBS, 0.5% BSA, 15 ppm Proclin) was added and incubated at room temperature (RT) for 1 hour. The plate was then washed three times with wash buffer and 25 μL of the neutralized peptide exchange reaction samples were transferred into the appropriate wells. The plate was incubated at RT for 2 hours and the unbound components were removed by washing the plate six times with wash buffer. The bound MHCII/peptide complex were then detected by adding 25 μL/well of horseradish peroxidase conjugated streptavidin (HRP-SA) in assay diluent (PBS 0.5% BSA, 0.05% Tween 20, 15 ppm Proclin, pH 7.4) at 25 ng/ml and incubated at RT for 1 hour. After removing the HRP-SA with 6 washes, the enzymatic reaction was developed with the peroxidase substrate tetramethylbenzidine (TMB, Moss Inc. cat #1000) and incubated at RT for 15 mins. The reaction was stopped with 25 μL/well of 1M phosphate acid and the absorbance was measured at 450 nm using reference 630 nM on a Multiscan spectrophotometer (Thermo Fisher). Sample containing HLA-DRB1/low affinity CLIP and biotinylated high affinity huCLIP without test peptide was used as the negative control for each allele. For ranking the binding affinity of peptide, IC₅₀ of each peptide was determined using the One-site—Fit logIC₅₀ model in GraphPad Prism 8.4.3.

To optimize the concentration of huCLIP-bio across different DRB1 alleles, huCLIP-bio were titrated to result final concentration range of 20,000 nM-0.02 nM in the reaction mix containing 100 nM of DRB1 alleles with low binding CLIP. The Bmax and Kd were determined using the curve fitting model of saturation specific binding with Hill slope in GraphPad Prism 8.4.3.

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the claims.

### SEQUENCES

TABLE 1
SEQ ID NOs: 1-33.

| SEQ ID NO: | Sequence |
|---|---|
| SEQ ID NO: 1 | PVSKARMATGALAQA |
| SEQ ID NO: 2 | PVSKMRMATGALAQA |
| SEQ ID NO: 3 | PVSKMRMATPLLMQA |
| SEQ ID NO: 4 | PVSQMRMATPLLMRP |
| SEQ ID NO: 5 | ISQVHAAHAEINEAGR |
| SEQ ID NO: 6 | PVSQARMATGALARP |
| SEQ ID NO: 7 | LPLKMLNIPSINVH |
| SEQ ID NO: 8 | HTYTIDWTKDAVTWS |
| SEQ ID NO: 9 | PKYVKQNTLKLAT |
| SEQ ID NO: 10 | EPDVYYTSAFVFPTK |
| SEQ ID NO: 11 | LGLLYTVKMYPNLNDL |
| SEQ ID NO: 12 | SPILRFLYANVGEEA |
| SEQ ID NO: 13 | YFESFVREFVATART |
| SEQ ID NO: 14 | IVYSLVTTISSLSRI |
| SEQ ID NO: 15 | DITYKVHLATPINSR |
| SEQ ID NO: 16 | MEVGWYRSPFSRVVHLYRNGK |
| SEQ ID NO: 17 | GGGGSLVPRGSGGGGS |
| SEQ ID NO: 18 | GGGGSENLYFQGGGGGS |
| SEQ ID NO: 19 | LPLQPFQLAFGHLVN |
| SEQ ID NO: 20 | LQPFQLAFGHLVNRQV |
| SEQ ID NO: 21 | FQLAFGHLVNRQVFRQ |
| SEQ ID NO: 22 | FGHLVNRQVFRQGPQ |
| SEQ ID NO: 23 | HYKYMCNSSCMGVMN |
| SEQ ID NO: 24 | YMCNSSCMGVMNRRP |
| SEQ ID NO: 25 | NSSCMGVMNRRPILT |
| SEQ ID NO: 26 | CMGVMNRRPILTIIT |
| SEQ ID NO: 27 | DIQMTQSPSSLSASVGDRVTITCRASQGIRNYLAAWYQQKP |
| | GKAPKLLIYAASTLQSGVPSRFSGSGSGTDFTLTISSLQPEDV |
| | ATYYCQRYNRAPYTFGQGTKVEIKRTVAAPSVFIF |
| SEQ ID NO: 28 | EVQLVESGGGLVQPGRSLRLSCAASGFTFDDYAMHWVRQA |
| | PGKGLEWVSAITWNSGHIDDYADSVEGRFTISRDNAKNSL |
| | YLQMNSLRAEDTAVYCAKVSYLSTASSLDYWGQGTLVTVS |
| | SASTKGPSV |
| SEQ ID NO: 29 | DIQMTQSPSSLSASV |
| SEQ ID NO: 30 | MTQSPSSLSASVGDR |
| SEQ ID NO: 31 | SPSSLSASVGDRVTI |
| SEQ ID NO: 32 | SLSASVGDRVTITC |
| SEQ ID NO: 33 | ASVGDRVTITCRASQ |

### REFERENCES

1. Yadav, M., et al. Predicting immunogenic tumor mutations by combining mass spectrometry and exome sequencing, Nature (2014) November 27; 515(7528):572-6.
2. Capietto, A. H., et al. Mutation position is an important determinant for predicting cancer neoantigens. J. Exp. Med. (2020) Apr. 6; 217(4):e20190179.

What is claimed is:

1. A composition comprising a test peptide and a major histocompatibility complex class II (MHCII)-ligand complex, the MHCII-ligand complex comprising: (i) an MHC molecule comprising an alpha chain and a beta chain, and (ii) a ligand associated with the alpha chain and the beta chain.

2. The composition of claim 1, wherein the alpha chain comprises a first dimerization tag and the beta chain comprises a second dimerization tag.

3. The composition of claim 2, wherein the dimerization tags interact with each other and stabilize association of the alpha chain and the beta chain.

4. The composition of claim 2 or 3, wherein the first dimerization tag and second dimerization tag comprise a dimerization pair selected from a leucine zipper, Fos/Jun, an iDimerize pair, coiled coil heterodimerization tag, or knob-in-hole or other heterodimeric Fcs.

5. The composition any one of claims 1 to 4, wherein the ligand is covalently bound to the alpha chain or the beta chain via a cleavable linker.

6. The composition of claim 5, wherein the cleavable linker is a UV-cleavable linker or an enzyme-cleavable linker.

7. The composition of any one of claims 1 to 6, further comprising a tagged peptide capable of binding the MHCII molecule and comprising a tag.

8. The composition of claim 7, further comprising a molecule that binds to the tag and comprises a detectable label.

9. The composition of claim 8, wherein the detectable label comprises a chemiluminescent compound or a fluorescent compound.

10. The composition of any one of claims 7 to 9, wherein the tag comprises biotin and the molecule that binds to the tag comprises streptavidin.

11. The composition of any one of claims 7 to 10, further comprising an antibody that binds to the MHCII molecule.

12. The composition of claim 11, wherein the antibody is immobilized to a surface.

13. The composition of any one of claims 1 to 12, wherein the ligand is produced at a level of at least about 1 mg/L, is detectable in 2D LCMS after cleavage of the linker; and has a peptide exchange yield of about 20% to 100% in an exchange reaction with a peptide known to have high affinity for the MHCII molecule.

14. The composition of any one of claims 7 to 13, wherein the tagged peptide has an affinity for the MHCII molecule that is higher than the affinity of the ligand for the MHCII molecule.

15. The composition of any one of claims 1 to 14, wherein the MHCII-ligand complex comprises an HLA peptide encoded by an HLA-DP, HLA-DM, HLA-DO, HLA-DQ, or HLA-DR allele.

16. The composition of claim 15, wherein the HLA-DR allele and the ligand are selected from:

| | HLA-DR allele | Ligand | SEQ ID NO. |
|---|---|---|---|
| a. | HLA-DRB1*01:01 | PVSKARMATGALAQA | SEQ ID NO.: 1 |
| b. | HLA-DRB1*03:01 | PVSKMRMATGALAQA | SEQ ID NO.: 2 |
| c. | HLA-DRB1*04:01 | PVSKMRMATGALAQA | SEQ ID NO.: 2 |
| d. | HLA-DRB1*07:01 | PVSKMRMATGALAQA | SEQ ID NO.: 2 |
| e. | HLA-DRB1*08:01 | PVSKMRMATPLLMQA | SEQ ID NO.: 3 |
| f. | HLA-DRB1*11:01 | PVSKMRMATGALAQA | SEQ ID NO.: 2 |
| g. | HLA-DRB1*13:01 | PVSKMRMATPLLMQA | SEQ ID NO.: 3 |
| h. | HLA-DRB1*15:01 | PVSKARMATGALAQA | SEQ ID NO.: 1 |
| i | HLA-DRB1*11:04 | PVSKMRMATGALAQA | SEQ ID NO.: 2 |

17. The composition of any one of claims 1 to 16, further comprising a MHCII-test peptide complex comprising: (i) an MHC molecule comprising an alpha chain and a beta chain, and (ii) a test peptide associated with the alpha chain and the beta chain.

18. The composition of any one of claims 1 to 17, further comprising a MHCII-tagged peptide complex comprising: (i) an MHC molecule comprising an alpha chain and a beta chain, and (ii) a tagged peptide associated with the alpha chain and the beta chain.

19. The composition of any one of claims 1 to 18, further comprising one or more additional test peptides.

20. The composition of any one of claims 1 to 19, wherein the test peptide and/or the one or more additional test peptides comprises a tumor antigen or neoantigen.

21. The composition of any one of claims 1 to 20, wherein the length of the test peptide and/or the one or more additional test peptides is between 7 amino acids and 30 amino acids.

22. A major histocompatibility complex class II (MHCII)-ligand complex comprising (i) an MHCII molecule comprising an alpha chain and a beta chain, and (ii) a ligand associated with the MHCII molecule.

23. The MHCII-ligand complex of claim 22, wherein the ligand comprises an amino acid sequence selected from: SEQ ID NO.: 1 to SEQ ID NO.: 16.

24. The MHCII-ligand complex of claim 22 or 23, wherein the ligand is bound to the alpha chain or the beta chain via a cleavable linker.

25. The MHCII-ligand complex of claim 24, wherein the cleavable linker is a UV-cleavable linker or an enzyme-cleavable linker.

26. The MHCII-ligand complex of any one of claims 22 to 25, wherein the alpha chain comprises a first dimerization tag and the beta chain comprises a second dimerization tag.

27. The MHCII-ligand complex of claim 26, wherein the dimerization tags interact with each other and stabilize association of the alpha chain and the beta chain.

28. The MHCII-ligand complex of claim 26 or 27, wherein the first dimerization tag and second dimerization tag comprise a dimerization pair selected from a leucine zipper, Fos/Jun, an iDimerize pair, coiled coil heterodimerization tag, or knob-in-hole or other heterodimeric Fcs.

29. The MHCII-ligand complex of any one of claims 22 to 25, wherein the MHCII molecule comprises an HLA peptide encoded by an HLA-DR allele, and the HLA-DR allele and the ligand are selected from:

| | HLA-DR allele | Ligand | SEQ ID NO. |
|---|---|---|---|
| a. | HLA-DRB1*01:01 | PVSKARMATGALAQA | SEQ ID NO.: 1 |
| b. | HLA-DRB1*03:01 | PVSKMRMATGALAQA | SEQ ID NO.: 2 |
| c. | HLA-DRB1*04:01 | PVSKMRMATGALAQA | SEQ ID NO.: 2 |
| d. | HLA-DRB1*07:01 | PVSKMRMATGALAQA | SEQ ID NO.: 2 |
| e. | HLA-DRB1*08:01 | PVSKMRMATPLLMQA | SEQ ID NO.: 3 |
| f. | HLA-DRB1*11:01 | PVSKMRMATGALAQA | SEQ ID NO.: 2 |
| g. | HLA-DRB1*13:01 | PVSKMRMATPLLMQA | SEQ ID NO.: 3 |
| h. | HLA-DRB1*15:01 | PVSKARMATGALAQA | SEQ ID NO.: 1 |
| i | HLA-DRB1*11:04 | PVSKMRMATGALAQA | SEQ ID NO.: 2 |

30. A method of detecting binding of a major histocompatibility complex class II (MHCII) molecule to a peptide, comprising:
a) providing a first composition comprising a peptide and a MHCII-ligand complex comprising (i) the MHCII molecule comprising an alpha chain and a beta chain and (ii) a ligand, wherein the ligand is bound to the alpha chain or the beta chain via a cleavable linker;
b) subjecting the first composition to a condition to cause cleavage of the cleavable linker;
c) incubating the first composition for a period of time sufficient to form a second composition, the second composition comprising the alpha chain, the beta chain, the ligand, free peptide, and/or a MHCII-peptide complex comprising the MHCII molecule and the peptide non-covalently bound to the MHCII molecule; and
d) determining whether the MHCII molecule is bound to the peptide.

31. The method of claim 30, wherein MHCII molecule binding to the peptide is determined by measuring a level of MHCII-peptide complex in the second composition.

32. The method of claim 30 or 31, wherein the level of MHCII-peptide complex is measured by 2-dimensional liquid chromatography-mass spectrometry (2D LC/MS) of the second composition.

33. The method of claim 32, wherein 2D LC/MS comprises removing the free peptide and/or ligand from the second composition.

34. The method of claim 33, further comprising high-performance liquid chromatography (HPLC) and mass spectrometry (MS) to distinguish the MHCII molecule and the peptide.

35. The method of claim 33 or 34, wherein the free peptide is removed from the second composition.

36. The method of claim 35, wherein the free peptide is removed from the second composition by size exclusion chromatography or ion exchange chromatography.

37. The method of any one of claims 33 to 36, wherein presence of the peptide as determined by HPLC and MS indicates that the MHCII molecule is capable of binding to the peptide.

38. The method of any one of claims 30 to 37, wherein the first composition comprises a plurality of the MHCII-ligand complex and at least two different peptides.

39. The method of claim 38, wherein the peptides are distinguished from each other by mass spectrometry based on the mass of each peptide in step d).

40. The method of any one of claims 30 to 39, wherein the peptide is present in the first composition at a ratio of at least 10:1 (peptide:MHCII molecule).

41. The method of any one of claims 30 to 40, wherein the cleavable linker is a UV-cleavable linker or an enzyme-cleavable linker.

42. The method of any one of claims 30 to 41, wherein the ligand is produced at a level of at least about 1 mg/L, is detectable in 2D LCMS after cleavage of the linker, and has a peptide exchange yield of about 20% to 100% in an exchange reaction with a peptide known to have high affinity for the MHCII molecule.

43. The method of any one of claims 30 to 42, wherein the MHCII molecule comprises an HLA peptide encoded by an HLA-DP, HLA-DM, HLA-DO, HLA-DQ, or HLA-DR allele.

44. The method of claim 43, wherein the HLA-DR allele and the ligand are selected from:

| | HLA-DR allele | Ligand | SEQ ID NO. |
|---|---|---|---|
| a. | HLA-DRB1*01:01 | PVSKARMATGALAQA | SEQ ID NO.: 1 |
| b. | HLA-DRB1*03:01 | PVSKMRMATGALAQA | SEQ ID NO.: 2 |
| c. | HLA-DRB1*04:01 | PVSKMRMATGALAQA | SEQ ID NO.: 2 |
| d. | HLA-DRB1*07:01 | PVSKMRMATGALAQA | SEQ ID NO.: 2 |
| e. | HLA-DRB1*08:01 | PVSKMRMATPLLMQA | SEQ ID NO.: 3 |
| f. | HLA-DRB1*11:01 | PVSKMRMATGALAQA | SEQ ID NO.: 2 |
| g. | HLA-DRB1*13:01 | PVSKMRMATPLLMQA | SEQ ID NO.: 3 |
| h. | HLA-DRB1*15:01 | PVSKARMATGALAQA | SEQ ID NO.: 1 |
| i | HLA-DRB1*11:04 | PVSKMRMATGALAQA | SEQ ID NO.: 2 |

45. The method of any one of claims 30 to 44, wherein the test peptide comprises a tumor antigen or neoantigen, or an autoantigen.

46. The method of any one of claims 30 to 45, wherein the length of the test peptide is between 7 amino acids and 30 amino acids.

47. A method for detecting the affinity of a test peptide for a major histocompatibility complex class II (MHCII) molecule, comprising:
a) providing a first composition comprising the test peptide; a tagged MHCII-binding peptide (tagged peptide); and a MHCII-ligand complex comprising (i) the MHCII molecule comprising an alpha chain and a beta chain and (ii) a ligand, wherein the ligand is bound to the alpha chain or the beta chain via a cleavable linker;
b) subjecting the first composition to a condition to cause cleavage of the cleavable linker;
c) incubating the first composition for a period of time sufficient form a second composition, the second composition comprising the alpha chain, the beta chain, the ligand, free test peptide, free tagged peptide, MHCII-tagged peptide complex comprising the tagged peptide associated with the MHCII molecule, and/or MHCII-test peptide complex comprising the test peptide associated with the MHCII molecule; and
d) determining whether the MHCII molecule is bound to the test peptide.

48. The method of claim 47, wherein the first composition is incubated for at least 24 hours after step b).

49. The method of claim 47, wherein the first composition is incubated for 24 hours to 96 hours after step b)

50. The method of any one of claims 47 to 49, further comprising contacting the second composition with an antibody that binds to said MHCII molecule.

51. The method of claim 50, wherein the antibody is bound to a solid surface.

52. The method of claim 50 or 51, further comprising determining an amount of MHCII-tagged peptide complex bound to the antibody.

53. The method of claim 52, wherein the tag comprises biotin and the determining step comprises contacting the MHCII-tagged peptide complex bound to the antibody with a streptavidin-horseradish peroxidase conjugate.

54. The method of claim 52 or 53, wherein the test peptide has a low affinity for the MHCII allele if the MHCII-tagged peptide complex is bound to the antibody.

55. The method of claim 52 or 53, wherein the test peptide has a high affinity for the MHCII allele if the MHCII-tagged peptide complex is not bound to the antibody.

56. The method of any one of claims 47 to 55, wherein the affinity of the test peptide for the MHCII molecule is confirmed by a peptide exchange assay.

57. The method of claim 56, wherein the peptide exchange assay comprises the method of any one of claims 30 to 41.

58. The method of any one of claims 47 to 57, wherein the tagged peptide comprises human CLIP.

59. The method of any one of claims 47 to 58, wherein the cleavable linker is a UV-cleavable linker or an enzyme-cleavable linker.

60. The method of any one of claims 47 to 59, wherein the test peptide is an antigen.

61. The method of claim 60, wherein the antigen is a tumor-associated antigen.

62. The method of claim 60, wherein the antigen is a neoantigen.

63. The method of any one of claims 47 to 62, wherein the MHCII molecule comprises an HLA peptide encoded by an HLA-DP allele, an HLA-DM allele, an HLA-DOA allele, an HLA-DOB allele, an HLA-DQ allele, or an HLA-DR allele.

64. The method of claim 63, wherein the HLA peptide is encoded by an HLA-DR allele, and the HLA-DR allele and the ligand are selected from:

| | HLA-DR allele | Ligand | SEQ ID NO. |
|---|---|---|---|
| a. | HLA-DRB1*01:01 | PVSKARMATGALAQA | SEQ ID NO.: 1 |
| b. | HLA-DRB1*03:01 | PVSKMRMATGALAQA | SEQ ID NO.: 2 |
| c. | HLA-DRB1*04:01 | PVSKMRMATGALAQA | SEQ ID NO.: 2 |
| d. | HLA-DRB1*07:01 | PVSKMRMATGALAQA | SEQ ID NO.: 2 |
| e. | HLA-DRB1*08:01 | PVSKMRMATPLLMQA | SEQ ID NO.: 3 |
| f. | HLA-DRB1*11:01 | PVSKMRMATGALAQA | SEQ ID NO.: 2 |
| g. | HLA-DRB1*13:01 | PVSKMRMATPLLMQA | SEQ ID NO.: 3 |
| h. | HLA-DRB1*15:01 | PVSKARMATGALAQA | SEQ ID NO.: 1 |
| i | HLA-DRB1*11:04 | PVSKMRMATGALAQA | SEQ ID NO.: 2 |

65. A method for multiplex epitope mapping for a major histocompatibility complex class II (MHCII) allele, comprising:
a) providing a first composition comprising a plurality of test peptides, and a plurality of MHCII-ligand complexes, each MHCII-ligand complex comprising (i) a MHCII molecule comprising an alpha chain and a beta chain and (ii) a ligand, wherein the ligand is bound to the alpha chain or the beta chain via a cleavable linker;
b) subjecting the first composition to a condition to cause cleavage of the cleavable linker;
c) incubating the first composition for a period of time sufficient to form a second composition, the second composition comprising the alpha chain, the beta chain, the ligand, free test peptide, and/or a plurality of MHCII-test peptide complexes, each MHCII-test peptide complex comprising the MHCII molecule and a test peptide non-covalently bound thereto; and
d) determining whether one or more of the plurality of test peptides is bound to the MHCII molecule in the second composition.

66. The method of claim 65, wherein the plurality of test peptides comprises overlapping peptides of an antigen.

67. The method of claim 66, wherein the antigen is a neoantigen, a tumor-associated antigen, or an autoantigen.

68. The method of any one of claims 65 to 67, wherein the length of each test peptide is between 7 amino acids and 30 amino acids.

69. The method of any one of claims 65 to 68, wherein MHCII molecule binding to the one or more of the plurality of test peptides is determined by measuring a level of each test peptide bound to the MHCII molecule in the second composition.

70. The method of claim 69, wherein the level of each test peptide bound to the MHCII molecule is measured by 2-dimensional liquid chromatography-mass spectrometry (2D LC/MS) of the second composition.

71. The method of claim 70, further comprising performing high-performance liquid chromatography (HPLC) and mass spectrometry (MS) to distinguish the MHCII molecule and the test peptides.

72. The method of any one of claims 65 to 71, wherein 2D LC/MS comprises removing the free test peptide and/or ligand from the second composition.

73. The method of claim 72, wherein the free test peptide is removed from the second composition by size exclusion chromatography or ion exchange chromatography.

74. The method of any one of claims 70 to 73, wherein presence of a given test peptide as determined by HPLC and MS indicates that the given test peptide is capable of binding to the MHCII molecule.

75. The method of any one of claims 70 to 73, wherein the relative levels of the test peptides bound to the MHCII molecules indicate the relative affinities of the test peptides for the MHCII molecule.

76. The method of claim 75, wherein a lower level of a first test peptide bound to the MHCII molecules compared to a second test peptide indicates that the first test peptide has a lower likelihood of binding the MHCII molecule on the surface of a cell than the second test peptide.

77. The method of claim 75 or 76, wherein a higher level of a second test peptide bound to the MHCII molecules compared to a first test peptide indicates that the second test peptide has a higher likelihood of binding the MHCII molecule on the surface of a cell than the first test peptide.

78. The method of any one of claims 70 to 78, wherein the test peptides are distinguished from each other by mass spectrometry based on the mass of each test peptide.

79. A peptide that binds at least one major histocompatibility complex class II (MHCII) molecule, the peptide comprising an amino acid sequence selected from: SEQ ID NO.: 1 to SEQ ID NO.: 16.
